(19) United States
(12) Patent Application Publication
(10) Pub. No.: US 2026/0224314 A1
(43) Pub. Date: Aug. 6, 2026

(54) MASTER-SLAVE SYSTEM, MASTER-SLAVE CONTROL DEVICE, AND MASTER-SLAVE CONTROL METHOD

(71) Applicant: SONY GROUP CORPORATION, TOKYO (JP)

(72) Inventors: HIROMASA MASUDA, TOKYO (JP); KAZUO HONGO, TOKYO (JP)

(21) Appl. No.: 19/157,489

(22) PCT Filed: Feb. 28, 2024
(86) PCT No.: PCT/JP2024/007449

§ 371 Date: Aug. 18, 2025

(30) Foreign Application Priority Data
Mar. 10, 2023 (JP) 2023-038091

(51) Int. Cl. A61B 34/37; A61B 34/00; A61B 34/30

(52) U.S. Cl. CPC A61B 34/37; A61B 34/25; A61B 2034/254; A61B 2034/301

(57) ABSTRACT

A master-slave system according to an embodiment of the present disclosure includes: a display unit that displays an image to a user who operates a master robot; and a display control unit that causes the display unit to display an image for locating a distal end of a slave robot in an interference avoidance region that has been preset.

### FIELD

The present disclosure relates to a master-slave system, a master-slave control device, and a master-slave control method.

### BACKGROUND

Various master-slave robots such as a master-slave type robot for endoscopic surgery have been developed. In a master-slave robot, it is important that a master-side motion from an operator is reproduced with high accuracy in a slave-side motion. In contrast, it is also important that the master-slave robot supports the operator and the human thereby safely performs a task using the master-slave robot. From such a viewpoint, in order to achieve a safe operation, for example, an interference detection technique and an interference avoidance technique of a robot have been proposed (e.g., see Patent Literatures 1 and 2.).

### CITATION LIST

### Patent Literature

Patent Literature 1: JP 2019-202354 A

Patent Literature 2: JP 2022-115645 A

### SUMMARY

### Technical Problem

Since, however, the above-described interference detection technique, interference avoidance technique, and the like need calculation for interference detection and interference avoidance, and the calculation takes time, a prompt slave motion is difficult to be achieved. Furthermore, since instructing a system in an interference prohibited area needs, for example, a complicated operation and time, prompt task start is difficult to be achieved.

Therefore, the present disclosure proposes a master-slave system, a master-slave control device, and a master-slave control method capable of achieving a prompt slave motion and prompt task start.

### Solution to Problem

A master-slave system according to an embodiment of the present disclosure includes: a display unit that displays an image to a user who operates a master robot; and a display control unit that causes the display unit to display an image for locating a distal end of a slave robot in an interference avoidance region that has been preset.

A master-slave control device according to an embodiment of the present disclosure causes a display unit that displays an image to a user who operates a master robot to display an image for locating a distal end of a slave robot in an interference avoidance region that has been preset.

A master-slave control method according to an embodiment of the present disclosure includes a computer causing a display unit that displays an image to a user who operates a master robot to display an image for locating a distal end of a slave robot in an interference avoidance region that has been preset.

### BRIEF DESCRIPTION OF DRAWINGS

FIG. 1 illustrates a configuration example of a master-slave system according to an embodiment of the present disclosure.

FIG. 2 illustrates a configuration example of a master device and a slave device according to the embodiment of the present disclosure.

FIG. 3 illustrates movable regions of a distal end of a slave robot in a case of an equal WS magnification according to the embodiment of the present disclosure.

FIG. 4 illustrates the movable regions of the distal end of the slave robot in a case of a WS magnification of 10 times according to the embodiment of the present disclosure.

FIG. 5 illustrates changes of the movable regions of the distal end of the slave robot in accordance with a WS magnification and a camera magnification according to the embodiment of the present disclosure.

FIG. 6 illustrates an interference avoidance region of the distal end of the slave robot according to the embodiment of the present disclosure.

FIG. 7 illustrates the interference avoidance region of the distal end of the slave robot according to the embodiment of the present disclosure.

FIG. 8 illustrates the interference avoidance region of the distal end of the slave robot according to the embodiment of the present disclosure.

FIG. 9 illustrates the interference avoidance region of the distal end of the slave robot according to the embodiment of the present disclosure.

FIG. 10 illustrates movement of the distal end of the slave robot into the interference avoidance region according to the embodiment of the present disclosure.

FIG. 11 illustrates the movement of the distal end of the slave robot into the interference avoidance region according to the embodiment of the present disclosure.

FIG. 12 illustrates the movement of the distal end of the slave robot into the interference avoidance region according to the embodiment of the present disclosure.

FIG. 13 illustrates the movement of the distal end of the slave robot into the interference avoidance region according to the embodiment of the present disclosure.

FIG. 14 illustrates a processing example of the movement of the distal end of the slave robot into the interference avoidance region according to the embodiment of the present disclosure.

FIG. 15 illustrates a processing example of the movement of the distal end of the slave robot into the interference avoidance region according to the embodiment of the present disclosure.

FIG. 16 illustrates a processing example of the movement of the distal end of the slave robot into the interference avoidance region according to the embodiment of the present disclosure.

FIG. 17 illustrates a processing example of the movement of the distal end of the slave robot into the interference avoidance region according to the embodiment of the present disclosure.

FIG. 18 illustrates a processing example of the movement of the distal end of the slave robot into the interference avoidance region according to the embodiment of the present disclosure.

FIG. 19 illustrates a processing example of a surgical tool replacement sequence according to the embodiment of the present disclosure.

FIG. 20 illustrates a processing example of the surgical tool replacement sequence according to the embodiment of the present disclosure.

FIG. 21 illustrates a processing example of the surgical tool replacement sequence according to the embodiment of the present disclosure.

FIG. 22 illustrates a processing example of the surgical tool replacement sequence according to the embodiment of the present disclosure.

FIG. 23 illustrates a processing example of the surgical tool replacement sequence according to the embodiment of the present disclosure.

FIG. 24 illustrates a processing example of the surgical tool replacement sequence according to the embodiment of the present disclosure.

FIG. 25 illustrates a processing example of the surgical tool replacement sequence according to the embodiment of the present disclosure.

FIG. 26 illustrates a processing example of the surgical tool replacement sequence according to the embodiment of the present disclosure.

FIG. 27 illustrates a processing example of the surgical tool replacement sequence according to the embodiment of the present disclosure.

FIG. 28 illustrates a processing example of the surgical tool replacement sequence according to the embodiment of the present disclosure.

FIG. 29 illustrates a processing example of the surgical tool replacement sequence according to the embodiment of the present disclosure.

FIG. 30 illustrates a processing example of the surgical tool replacement sequence according to the embodiment of the present disclosure.

FIG. 31 illustrates a processing example of the surgical tool replacement sequence according to the embodiment of the present disclosure.

FIG. 32 illustrates a processing example of the surgical tool replacement sequence according to the embodiment of the present disclosure.

FIG. 33 illustrates a configuration example of hardware according to the embodiment of the present disclosure.

### DESCRIPTION OF EMBODIMENTS

An embodiment of the present disclosure will be described in detail below with reference to the drawings. The embodiment also includes examples and variations. Note that the embodiment does not limit a system, a device, a method, and the like according to the present disclosure. Furthermore, in the following embodiment, basically, the same reference signs are attached to the same portions, and duplicate description is omitted.

One or a plurality of embodiments below can each be implemented independently. In contrast, at least a part of the plurality of embodiments below may be appropriately combined with at least a part of other embodiments to be implemented. The plurality of embodiments can include different novel features. Therefore, the embodiments can contribute to achieving different objects or solving different problems, and exhibit different effects.

The present disclosure will be described in the following item order.
1. Embodiment
1-1. Configuration Example of Master-Slave System
1-2. Movable Regions of Distal End of Slave Robot
1-3. Interference Avoidance Region of Distal End of Slave Robot
1-4. Movement of Distal End of Slave Robot into Interference Avoidance Region
1-5. Processing Example of Movement of Distal End of Slave Robot into Interference Avoidance Region
1-6. Processing Example of Surgical Tool Replacement Sequence
1-7. Effects
2. Other Embodiments
3. Configuration Example of Hardware
4. Appendix

### 1. Embodiment

### 1-1. Configuration Example of Master-Slave System

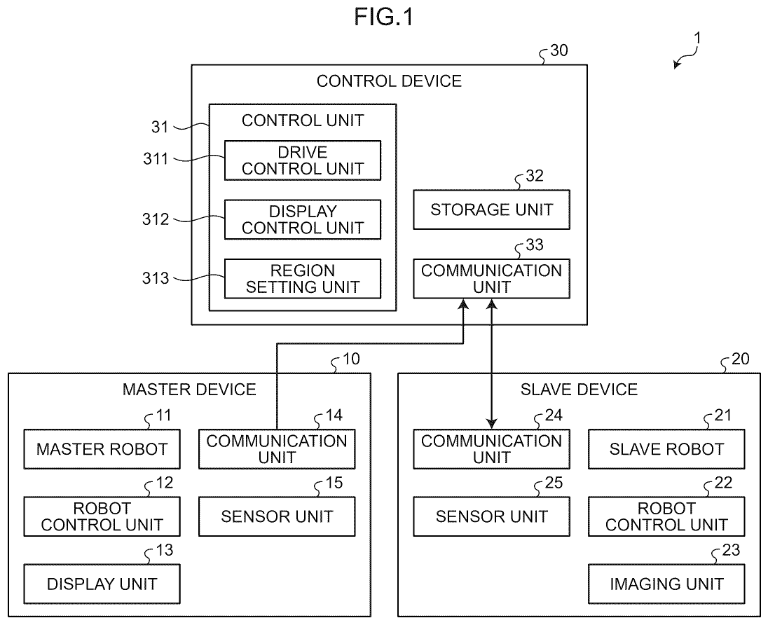

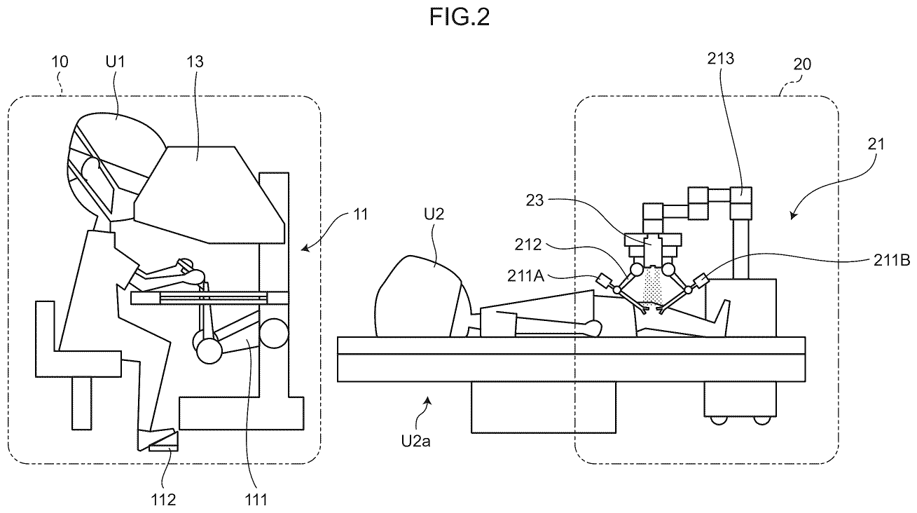

A configuration example of a master-slave system 1 according to the embodiment will be described with reference to FIGS. 1 and 2. FIG. 1 illustrates a configuration example of the master-slave system 1 according to the embodiment. FIG. 2 illustrates a configuration example of a master device 10 and a slave device 20 according to the embodiment. The master-slave system 1 uses a master-slave type robot (master-slave robot).

As illustrated in FIG. 1, the master-slave system 1 includes the master device 10, the slave device 20, and a control device 30. The control device 30 is communicably connected to each of the master device 10 and the slave device 20. Various types of information are transmitted and received between the devices. The transmission and reception may be executed via, for example, various communication networks in one or both of a wireless manner and a wired manner.

### (Master Device)

The master device 10 includes a master robot 11, a robot control unit 12, a display unit 13, a communication unit 14, and a sensor unit 15. For example, the master device 10 has a function of controlling the drive of the slave device 20 and a function of presenting information from the slave device 20 to a user.

The master robot 11 functions as an input device for a user such as a surgeon to perform a remote operation on the slave device 20 mounted with surgical tools such as forceps. The master robot 11 has a configuration suitable for the user to remotely operate the slave device 20. The master robot 11 moves based on a drive signal from the robot control unit 12.

The robot control unit 12 controls the position of the master robot 11 in accordance with a value of a position command designated by the user through the master robot 11 or the like. Examples of the controlled position include the position of a distal end (arm tip position) of the master robot 11. The control is performed based on information designating the position of the distal end (arm tip position command). The arm tip position command is generated in response to a user operation. The control device 30 generates the arm tip position command under bilateral control. The robot control unit 12 controls, for example, the rotation (e.g., rotation speed, rotation angular speed, and torque) of a joint portion so that the distal end of the master robot 11 is located at a position in accordance with the arm tip position command.

The display unit 13 displays various images. The display unit 13 presents information on work being performed by the slave device 20 to the user operating the master robot 11 mainly based on image information acquired by the slave device 20. Examples of the display unit 13 include an installation type display and a head mounted display (HMD) mounted on the head of the user.

The communication unit 14 enables communication of various types of information with the control device 30. For example, the communication unit 14 transmits input information to the master robot 11 and sensor information obtained from the sensor unit 15 to the control device 30. Furthermore, the communication unit 14 receives information (e.g., control information and various types of information on side of slave device 20) transmitted from the control device 30.

The sensor unit 15 detects the state of the master robot 11. Examples of the detected state include a torque reference value, an angle (joint angle), and an angular speed (joint angular speed) of a joint portion. These pieces of information are used as the sensor information. The torque reference value substantially corresponds to a current value input to the master robot 11, and can be detected by the master device 10. The joint angle is acquired from, for example, an encoder in an actuator provided in the joint portion of the master robot 11. The joint angular speed is acquired by performing time differentiation on the joint angle. Other examples of the detected state include an acceleration reference value, a position, and a speed of the distal end input to the master robot 11. The above-described torque reference value is based on the acceleration reference value. The master device 10 can detect the acceleration reference value. Since identified from, for example, the above-described joint angle and joint angular speed, the position of the distal end and the speed of the distal end can be detected.

### (Slave Device)

The slave device 20 includes a slave robot 21, a robot control unit 22, an imaging unit 23, a communication unit 24, and a sensor unit 25. For example, the slave device 20 includes a mechanism driven by an actuator such as a motor, and moves under drive control from the master device 10. Furthermore, the slave device 20 has a function of presenting, to the master device 10, force and vibration at the time when a work target and a portion of the slave device 20, which is to come into contact with the target, are brought into contact with each other.

The slave robot 21 is remotely operated by the user such as a surgeon. For example, the slave robot 21 is an arm type robot having an articulated link structure, and mounted with a work unit serving as an end effector at a distal end of the robot. The slave robot 21 moves based on a drive signal from the robot control unit 22.

The robot control unit 22 controls the position of the slave robot 21 in accordance with a value of a position command designated by the user. Examples of the controlled position include the position of a distal end (arm tip position) of the slave robot 21. The control is performed based on information designating the position of the distal end (arm tip position command). The arm tip position command is generated in response to a user operation. The control device 30 generates the arm tip position command under bilateral control. The robot control unit 22 controls, for example, the rotation (e. g., rotation speed, rotation angular speed, and torque) of a joint portion so that the distal end of the slave robot 21 is located at a position in accordance with the arm tip position command.

The imaging unit 23 acquires an image of a work range of the target by imaging. For example, the imaging unit 23 has a zoom mechanism, and can change an imaging magnification (zoom magnification). The control device 30 can control the zoom magnification of the imaging unit 23. Note that, although the imaging unit 23 is provided in the slave robot 21, the position and posture of the imaging unit 23 may be changed by the imaging unit 23 being supported by a robot arm other than a slave arm 212 and the angle of a joint of the robot arm and the like being controlled by the robot control unit 22, for example. Examples of the imaging unit 23 include an RGB camera and a stereo camera.

The communication unit 24 enables communication of various types of information with the control device 30. For example, the communication unit 24 receives information (e.g., control information and various types of information on side of master device 10) transmitted from the control device 30. Furthermore, the communication unit 24 transmits sensor information obtained from the sensor unit 25 to the control device 30.

The sensor unit 25 detects the state of the slave robot 21. Examples of the detected state include a torque reference value, an angle (joint angle) , and an angular speed (joint angular speed) of a joint portion. These pieces of information are used as the sensor information. The torque reference value substantially corresponds to a current value input to the slave robot 21, and can be detected by the slave device 20. The joint angle is acquired from, for example, an encoder in an actuator provided in the joint portion of the slave robot 21. The joint angular speed is acquired by performing time differentiation on the joint angle. Other examples of the detected state include an acceleration reference value, a position, and a speed of the distal end input to the slave robot 21. The above-described torque reference value is based on the acceleration reference value. The slave device 20 can detect the acceleration reference value. Since identified from, for example, the above-described joint angle and joint angular speed, the position of the distal end and the speed of the distal end can be detected.

### (Control Device)

The control device 30 includes a control unit 31, a storage unit 32, and a communication unit 33. The control unit 31 includes a drive control unit 311, a display control unit 312, and a region setting unit 313.

The drive control unit 311 controls each unit related to the drive included in the master-slave system 1. For example, the drive control unit 311 acquires sensor information transmitted from the master device 10, and acquires a master position, which is the position of the master robot 11, based on the acquired sensor information. Then, the drive control unit 311 controls a slave position, which is the position of the slave robot 21, based on the acquired information such as the master position.

For example, the drive control unit 311 controls the master robot 11 and the slave robot 21 so that the position of the master robot 11 corresponds to the position of the slave robot 21. The correspondence between the positions here means that the positions of the corresponding portions of the master robot 11 and the slave robot 21 have a correspondence relation with each other. For example, the master robot 11 and the slave robot 21 are controlled so that the position of the distal end of the master robot 11 and the position of the distal end of the slave robot 21 have a correspondence relation.

Furthermore, the drive control unit 311 controls the master robot 11 and the slave robot 21 so that external force of the master robot 11 corresponds to external force of the slave robot 21. The correspondence between the external forces here means that the external forces of the corresponding portions of the master robot 11 and the slave robot 21 have a correspondence relation with each other. For example, the master robot 11 and the slave robot 21 are controlled so that the external force of the distal end of the master robot 11 and the external force of the distal end of the slave robot 21 have a correspondence relation.

The display control unit 312 mainly controls each unit related to a video included in the master-slave system 1. For example, the display control unit 312 acquires the image information transmitted from the slave device 20, and controls the display unit 13 and the like of the master device 10 based on the acquired image information. In the case, the display control unit 312 generates various images based on the image information, and sends the generated various images to the display unit 13.

The region setting unit 313 sets an interference avoidance region of the slave robot 21. The interference avoidance region is, for example, a region where the distal end of the slave robot 21 can be avoided from interfering with a surrounding environment such as a user and a bed. The interference avoidance region is preset. The interference avoidance region as described above will be described in detail later.

The storage unit 32 stores various types of information. For example, the storage unit 32 stores various types of information such as control information, sensor information, and image information. These pieces of information are transmitted from one or both of the master device 10 and the slave device 20, for example. Furthermore, the storage unit 32 also stores various types of information (e.g., program) necessary for processing executed in the control device 30.

The communication unit 33 enables communication of various types of information with the master device 10 and the slave device 20. For example, the communication unit 33 receives information (e.g., various types of information on side of master device 10 and various types of information on side of slave device 20) transmitted from the master device 10 and the slave device 20. Furthermore, the communication unit 33 transmits various types of information (e.g., control information, sensor information, and image information) to the master device 10 and the slave device 20.

As illustrated in FIG. 2, the master-slave system 1 as described above is used in, for example, surgery. In an example of FIG. 2, a surgeon (e.g., doctor) who performs surgery is illustrated as a user U1, and a patient who undergoes surgery is illustrated as a user U2. The user U2 is lying on a bed U2a, for example.

As illustrated in FIG. 2, the user U1 performs surgery on the user U2 by operating the master device 10 and remotely operating the slave device 20. Specifically, the master device 10 includes the master robot 11 operated by the user U1. The slave device 20 includes the slave robot 21 remotely operated by the user U1. Note that, although, in the embodiment, the master robot 11 and the slave robot 21 are medical robots, the master robot 11 and the slave robot 21 may be industrial robots in an industrial field other than a medical field.

The master robot 11 has a configuration suitable for the user U1 to remotely operate the slave robot 21. The illustrated master robot 11 holds a master arm 111, the display unit 13, and the like. The master arm 111 is operated by the user U1. The content of an operation of the master robot 11 performed by the user U1 (user operation) is transmitted from the master device 10 to the slave device 20 via the control device 30 as control information (input information). Furthermore, the master robot 11 has a configuration that supports the slave robot 21 so as to transmit the state of the slave robot 21 to the user U1, for example. The master robot 11 may include, for example, an arm similar to that of the slave robot 21. The user Ul can recognize the state of the slave robot 21 via the master robot 11. Note that a foot switch 112 may be provided on a floor in front of the master robot 11. When the foot switch 112 is pressed, a signal is transmitted from the master device 10 to the control device 30.

The master arm 111 is operated by both the right hand and the left hand of the user U1, for example. The user U1 places both arms or both elbows on a support base, and grips the master arm 111 with each of the right hand and the left hand. In this state, the user U1 operates the master arm 111 while viewing the display unit 13 on which a surgical field is displayed. For example, the user U1 remotely operates the position or orientation of a surgical tool attached to the slave device 20 or remotely operates a gripping motion through surgical tools by displacing the position and orientation of the master arm 111. The master arm 111 can transmit, to the user U1, a feel of contact of a surgical tool of the slave device 20 with, for example, an affected area of the patient.

The slave robot 21 includes, for example, an arm. The illustrated slave robot 21 includes a plurality of surgical tool units 211A and 211B, the slave arm 212, and a base arm 213. Although the slave robot 21 has, for example, six degrees of freedom of a distal end position and a posture, the number of degrees of freedom of the robot is not particularly limited. The slave arm 212 holds each of the surgical tool units 211A and 211B. In the example of FIG. 2, two surgical tool units are provided. Each of the slave arm 212 and the base arm 213 includes, for example, a plurality of link portions and a plurality of joint portions. The base arm 213 holds, for example, the slave arm 212 and the imaging unit 23. Note that examples of individual surgical tools of the surgical tool units 211A and 211B include forceps, tweezers, scissors, a pneumoperitoneum tube, an energy treatment tool, and a retractor.

Here, in the master device 10, an operation command for remotely operating the slave robot 21 is input via the master robot 11. The operation command includes, for example, a panning motion and a tilting motion of the slave arm 212 of the slave robot 21, a rotating motion about individual longitudinal axes (or roll axes) of the surgical tool units 211A and 211B, and motions of individual surgical tool distal ends of the surgical tool units 211A and 211B. The control device 30 generates a control command based on the received operation command, and transmits the control command to the slave device 20. The slave device 20 controls the drive of the slave robot 21 so as to achieve a motion of the slave robot 21 in accordance with the received control command.

For example, in the master device 10, instructions to perform a motion of the slave robot 21 and a motion of a surgical tool (e.g., yaw motion, pitch motion, and opening/closing motion of forceps) are given via the master robot 11. For example, when receiving an operation command giving an instruction to perform the yaw motion, the pitch motion, and the opening/closing motion of the forceps, the control device 30 calculates a rotation angle of each motor of the slave robot 21 and generates an angle command to each motor in order to achieve a motion of the target forceps. A control command including the angle command and the like is transmitted to the slave device 20.

### 1-2. Movable Regions of Distal End of Slave Robot

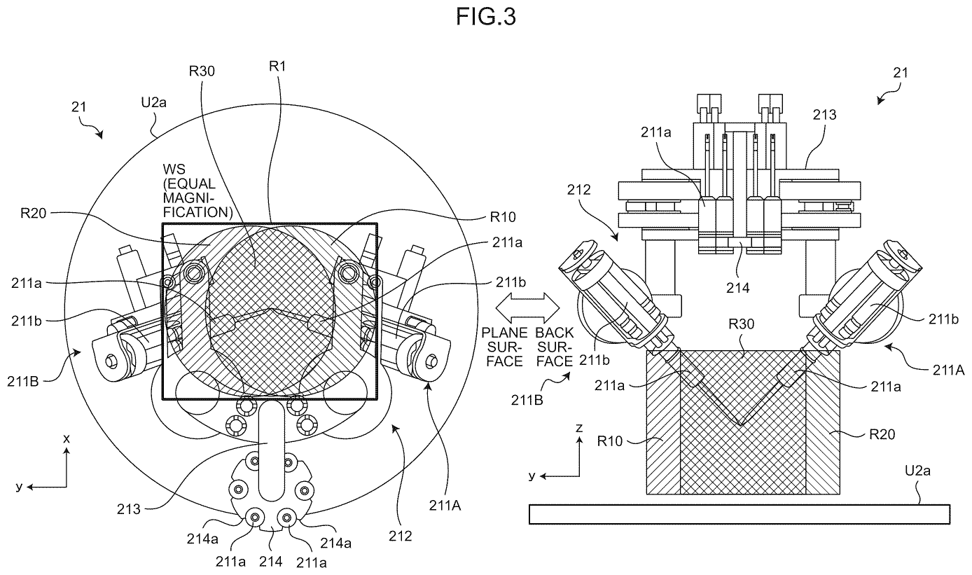

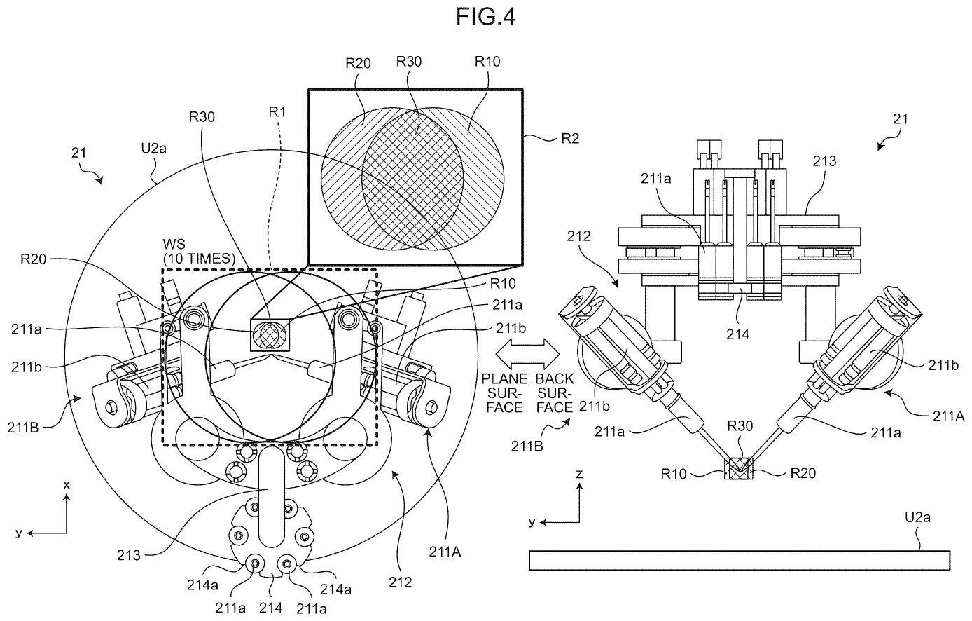

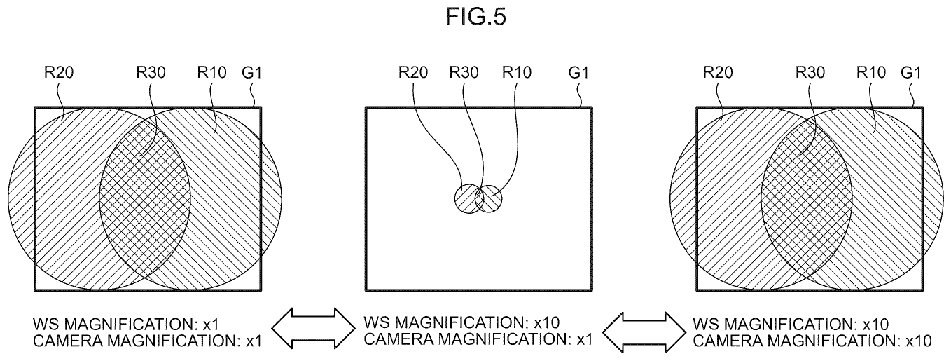

Movable regions (movable ranges) R10, R20, and R30 of the distal end of the slave robot 21 according to the embodiment will be described with reference to FIGS. 3 to 5. FIG. 3 illustrates the movable regions R10, R20, and R30 of the distal end of the slave robot 21 in a case of an equal workstation (WS) magnification according to the embodiment. FIG. 4 illustrates the movable regions R10, R20, and R30 of the distal end of the slave robot 21 in a case of a WS magnification of 10 times according to the embodiment. FIG. 5 illustrates changes of the movable regions R10, R20, and R30 of the distal end of the slave robot 21 in accordance with a WS magnification and a camera magnification according to the embodiment.

Note that the distal end of the slave robot 21 includes individual distal ends of the surgical tool units 211A and 211B (specifically, distal ends of individual surgical tools 211a of surgical tool units 211A and 211B) of the slave arm 212. The WS magnification corresponds to an operation magnification.

As illustrated in FIGS. 3 and 4, a visual field R1 of the imaging unit 23 includes: the movable region R10 of the distal end of the surgical tool unit 211A; a movable region R20 of the distal end of the surgical tool unit 211B; and the common movable region R30 in which the movable regions R10 and R20 overlap each other. The common movable region R30 is an interference region where the surgical tool units 211A and 211B interfere with each other. In an example of FIG. 3, an equal magnification is adopted (WS=equal magnification), and the movable regions R10, R20, and R30 are illustrated in the sizes of the equal magnification. The movable regions R10, R20, and R30 in an image displayed by the display unit 13 have individual sizes of an equal magnification. In contrast, in an example of FIG. 4, a magnification of 10 times (WS=10 times) is adopted, and the movable regions R10, R20, and R30 are illustrated in the sizes of 1/10 in the example of FIG. 3. The movable regions R10, R20, and R30 in an image R2 displayed by the display unit 13 have individual sizes of 10 times of actual sizes (enlarged display).

Here, each of the surgical tool units 211A and 211B includes a surgical tool 211a and a drive unit 211b. The surgical tool 211a can be detached from the drive unit 211b. Therefore, the surgical tool 211a can be replaced in each of the surgical tool units 211A and 211B. Furthermore, the slave robot 21 is provided with a surgical tool stand 214. The surgical tool stand 214 can individually store a plurality of surgical tools 211a. In the example of FIG. 3, the surgical tool stand 214 has a circular shape in plan view. A plurality of storage portions 214a is formed in an outer peripheral portion of the circular surgical tool stand 214. The storage portions 214a are formed by cutting out a plurality of outer peripheral portions of the circular surgical tool stand 214. The surgical tool 211a is stored in each of these storage portions 214a. For example, the surgical tool 211a stored in the surgical tool stand 214 and the surgical tool 211a of one or both of the surgical tool units 211A and 211B are automatically replaced with each other by a motion of the slave arm 212.

As illustrated in FIG. 5, in a display image G1, the individual sizes of the movable regions R10, R20, and R30 change between a case of a WS magnification of 1 time and a camera magnification of 1 time, a case of a WS magnification of 10 times and a camera magnification of 1 time, and a case of a WS magnification of 10 times and a camera magnification of 10 times, for example. Such a WS magnification and a camera magnification are changed in accordance with a work situation. For example, the user Ul changes the WS magnification and the camera magnification. The WS magnification corresponds to the operation magnification of the display unit 13, and the camera magnification corresponds to the magnification of the imaging unit 23.

In the master-slave system 1, work on a finer region can be done while enlarging the finer region by dynamically setting the operation magnification, that is, making a change in the operation magnification possible, for example, setting the operation magnification to be larger. Since the master robot 11 has a constant movable region regardless of an operation magnification, however, an operation magnification set to be larger makes an operation region smaller, which may make a large motion difficult (see FIG. 4). Furthermore, when the operation magnification is set to be larger, a larger and quicker operation in the master robot 11 is necessary for a large and quick motion in the slave robot 21. Such a motion may be made difficult. Therefore, in order to simultaneously make a minute motion and a large motion requiring speed in the slave robot 21, an operation magnification can be desirably changed seamlessly.

For example, in laparoscopic surgery using the master-slave system 1, an operation is often performed in a narrow magnification range at a relatively low operation magnification. In contrast, in a field of, for example, brain surgery, an operation is performed in a wide magnification range at a relatively high operation magnification. In such a field, the capability of easily change an operation magnification is particularly important. For example, in brain surgery, a precise operation is required to be performed at a relatively high operation magnification when sutures are connected. In contrast, a larger motion is required to pull the connected sutures. Furthermore, when a medical surgical tool is changed, it is necessary to move the surgical tool more greatly and separate the surgical tool once from an affected area. In addition, in order to avoid contact with another portion, it is necessary to lower an operation magnification and check the surrounding situation.

As described above, the operation magnification is changed by various types of work. Interference caused by various tasks is required to be avoided in a motion of the slave robot 21, that is, the slave arm 212 in the master-slave system 1. Examples of the interference include the interference between each of the surgical tool units 211A and 211B and the user U2 on the bed U2a and the interference between the surgical tool units 211A and 211B. In the case, calculation necessary for avoiding interference at the time of task execution is omitted by creating a situation, in which the interference can be avoided, before the task execution without avoiding the interference at the time of task execution. This can achieve an effect of safe and prompt task execution. The embodiment having such an effect will be described in detail below.

Note that examples of the task include a surgical tool replacement task. In the surgical tool replacement task, the slave robot 21 can automatically replace the individual surgical tools 211a of the surgical tool units 211A and 211B. It is important to avoid, for example, the interference between each of the surgical tool units 211A and 211B and the user U2 on the bed U2a and the interference between the surgical tool units 211A and 211B before executing the replacement operation. During surgery, the surgical tools 211a are usually changed many times. For example, a plurality of portions (e.g., four portions) of the user U2 may be treated in one piece of surgery. Surgery for one portion has a workflow of treatments including vessel search, anastomosis preparation, and anastomosis. In such surgery, the surgical tools 211a are changed many times. For example, various surgical tools such as forceps, tweezers, and scissors are replaced many times. Replacement time in the case is required to be, for example, approximately ten and several seconds such as 15 seconds.

### 1-3. Interference Avoidance Region of Distal End of Slave Robot

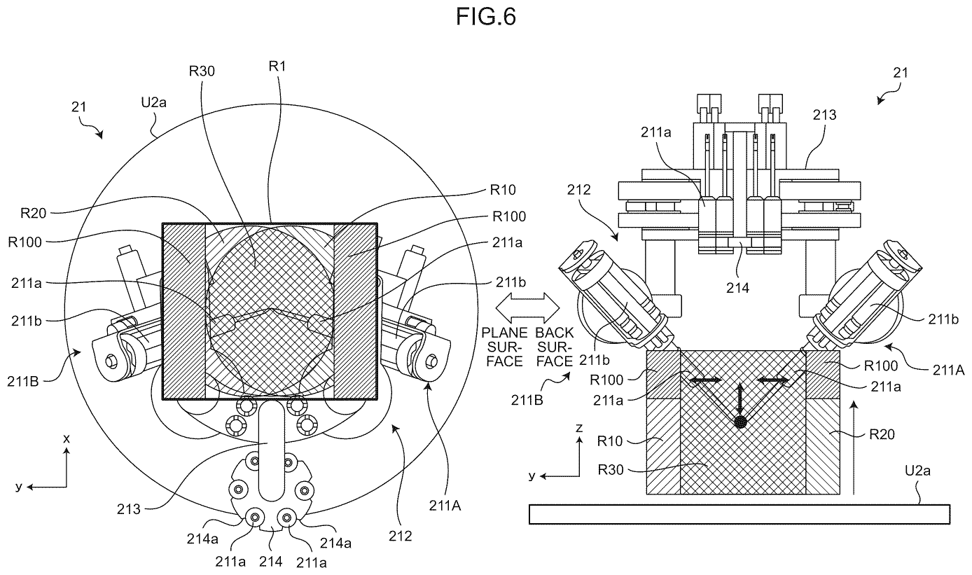

An interference avoidance region (interference avoidance range) R100 of the distal end of the slave robot 21 according to the embodiment will be described with reference to FIGS. 6 to 9. FIGS. 6 to 9 illustrate the interference avoidance region R100 of the distal end of the slave robot 21 according to the embodiment.

As illustrated in FIG. 6, the region setting unit 313 sets the interference avoidance region R100. In the interference avoidance region R100, the individual distal ends of the surgical tool units 211A and 211B do not interfere with the user U2 on the bed U2a, and do not interfere with each other. That is, the interference avoidance region R100 is a region where the interference between both the arms and the interference between both the arms and the user U2 are avoided. For example, the interference avoidance region R100 is a region where the height positions (positions in Z direction in FIG. 6) of the individual distal ends of the surgical tool units 211A and 211B are equal to or more than a predetermined threshold in the movable regions R10 and R20 of the individual distal ends of the surgical tool units 211A and 211B and the movable region R30 common to the individual distal ends of the surgical tool units 211A and 211B is excluded. For example, the predetermined threshold is preset, and the interference avoidance region R100 is also preset. The predetermined threshold and the interference avoidance region R100 are preliminarily determined theoretically or experimentally based on, for example, the motion of the slave robot 21 (e.g., slave arm 212 and base arm 213).

Note that, although, in an example of FIG. 6, the interference avoidance region R100 is set for each of the individual distal ends of the surgical tool units 211A and 211B, this is not a limitation. For example, when the slave robot 21 holds only one of the surgical tool units 211A and 211B, the interference avoidance region R100 may be set for only one of the surgical tool units 211A and 211B.

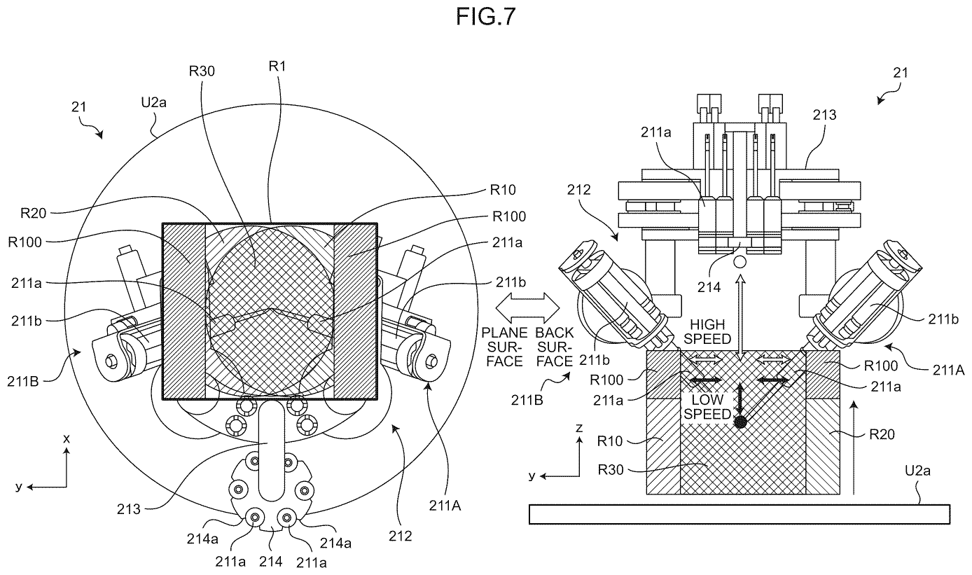

As illustrated in FIG. 7, the region setting unit 313 sets the interference avoidance region R100 as in the example of FIG. 6. Furthermore, the drive control unit 311 sets moving speeds of the individual distal ends of the surgical tool units 211A and 211B in accordance with the height positions of the individual distal ends of the surgical tool units 211A and 211B. In an example of FIG. 7, when the height positions of the individual distal ends of the surgical tool units 211A and 211B are lower than a predetermined height position, moving speeds of the individual distal ends of the surgical tool units 211A and 211B are set to be low. When the height positions of the individual distal ends of the surgical tool units 211A and 211B are equal to or higher than the predetermined height position, the moving speeds of the individual distal ends of the surgical tool units 211A and 211B are set to be high. That is, the moving speeds of the individual distal ends of the surgical tool units 211A and 211B in the case where the height positions of the individual distal ends of the surgical tool units 211A and 211B are equal to or higher than the predetermined height position is set to be higher than the moving speeds of the individual distal ends of the surgical tool units 211A and 211B in the case where the height positions of the individual distal ends of the surgical tool units 211A and 211B are lower than the predetermined height position. The predetermined height positions may be the same as or different from the above-described predetermined threshold. For example, the predetermined height position is a height position equal to or higher than the above-described predetermined threshold.

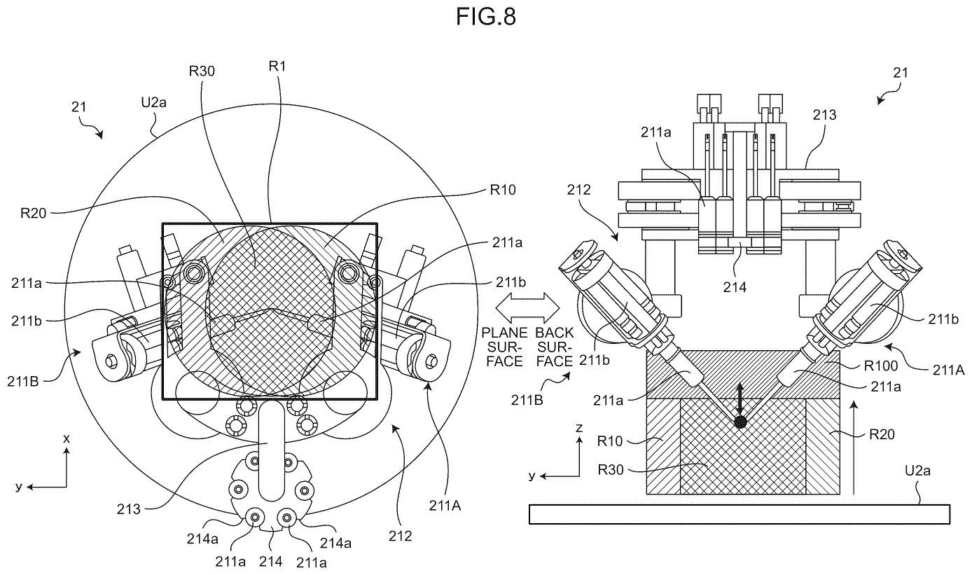

Although, as illustrated in FIG. 8, the region setting unit 313 sets the interference avoidance region R100 as in the example of FIG. 6, the interference avoidance region R100 includes only a region, where the height positions of the individual distal ends of the surgical tool units 211A and 211B are equal to or higher than the predetermined threshold, in the movable regions R10 and R20 of the individual distal ends of the surgical tool units 211A and 211B unlike in the example of FIG. 6. That is, the interference avoidance region R100 is not limited to the region in the example of FIG. 6, and may be, for example, a region in an example of FIG. 8.

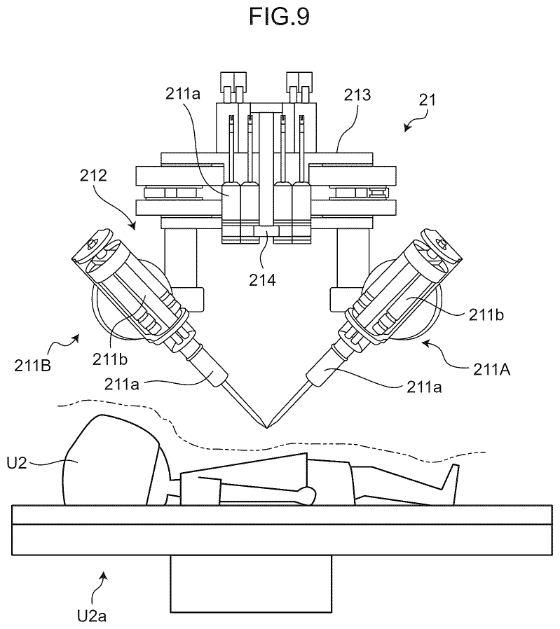

As illustrated in FIG. 9, the above-described predetermined threshold in a height direction may be set based on a predetermined separation distance between the individual distal ends of the surgical tool units 211A and 211B and the user U2 on the bed U2a. The predetermined threshold in the height direction is determined and set before surgery. For example, the slave robot 21 moves the individual distal ends of the surgical tool units 211A and 211B along the user U2 on the bed U2a without bringing the distal ends into contact with the user U2. The predetermined threshold in the height direction is thereby determined before surgery. In the case, the drive control unit 311 automatically moves the individual distal ends of the surgical tool units 211A and 211B so that the above-described predetermined separation distance is maintained.

### 1-4. Movement of Distal End of Slave Robot into Interference Avoidance Region

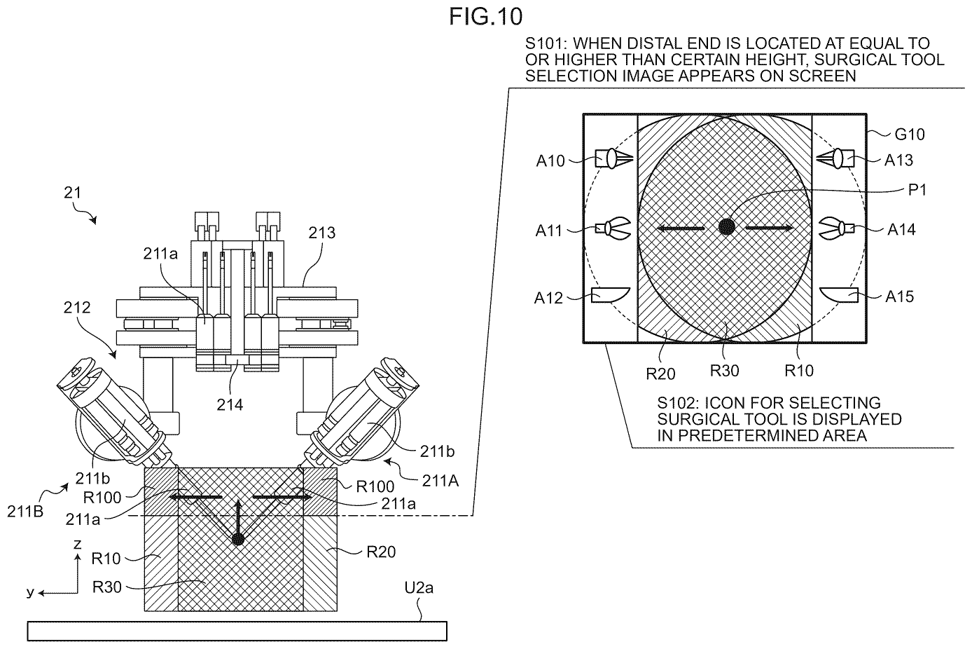

Movement of the distal end of the slave robot 21 into the interference avoidance region R100 according to the embodiment (movements of individual distal ends of surgical tool units 211A and 211B of slave robot 21) will be described with reference to FIGS. 10 to 13. FIGS. 10 to 13 illustrate the movement of the distal end of the slave robot 21 into the interference avoidance region R100 according to the embodiment.

### (Manual Movement of Distal End of Slave Robot 21)

As illustrated in FIG. 10, in Step S101, when the individual distal ends of the surgical tool units 211A and 211B of the slave robot 21 are located at positions equal to or higher than a certain height (predetermined threshold), a surgical tool selection image G10 appears on a screen of the display unit 13. The surgical tool selection image G10 is used for guiding the distal end of the slave robot 21, that is, the individual distal ends of the surgical tool units 211A and 211B into the preset interference avoidance region R100. The surgical tool selection image G10 is, for example, a user interface (UI) image. The surgical tool selection image G10 is displayed at the time of shift of the slave robot 21 to an autonomous motion task.

Note that the surgical tool selection image G10 is an example of a tool selection image. The surgical tool 211a is an example of a tool. Since, in the embodiment, the master robot 11 and the slave robot 21 are medical robots, the tool is the surgical tool 211a, and a tool selection screen is the surgical tool selection image G10. When the master robot 11 and the slave robot 21 are industrial robots, however, the tool and the tool selection screen may have other names.

In Step S102, a plurality of icons A10 to A15 for selecting a surgical tool is displayed in a predetermined area on the surgical tool selection image G10. Each of the icons A10 to A15 is an image of a graphic representing the surgical tool 211a, for example. Examples of the surgical tool 211a include forceps, tweezers, scissors, and energy treatment tools. Furthermore, a pointer P1 is also displayed on the surgical tool selection image G10. The pointer Pl corresponds to the distal end of the surgical tool unit 211A, and moves in a corresponding direction and movement amount in accordance with the movement of the distal end of the surgical tool unit 211A.

The above-described predetermined area is arranged at a position corresponding to the interference avoidance region R100. In an example of FIG. 10, the predetermined area is provided at both ends of the surgical tool selection image G10 in accordance with the interference avoidance region R100. The right predetermined area corresponds to the distal end of the surgical tool unit 211A. The left predetermined area corresponds to the distal end of the surgical tool unit 211B.

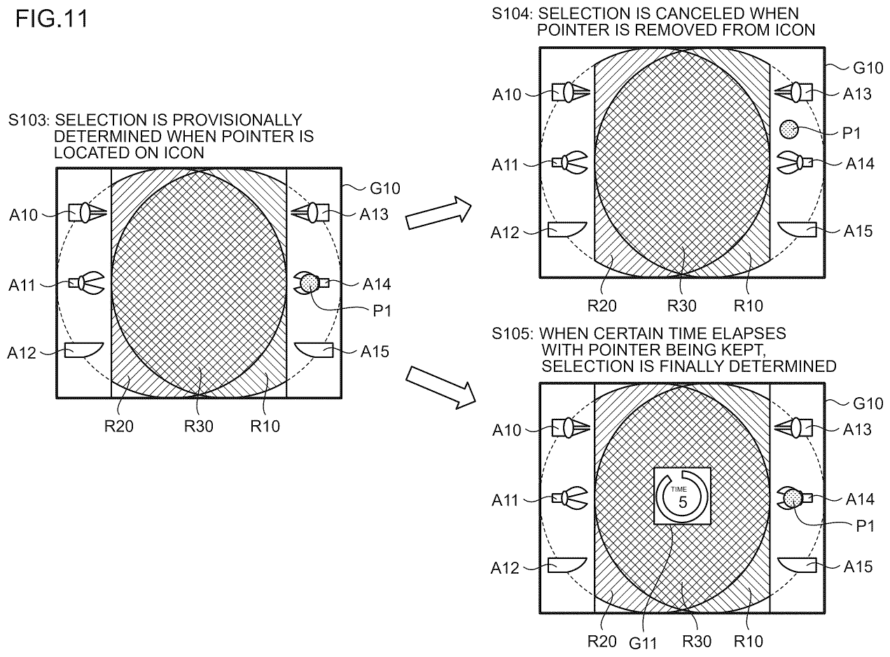

As illustrated in FIG. 11, in Step S103, when the pointer P1 is located on one of the icons A10 to A15, for example, when the pointer P1 is located on the icon A14, the surgical tool 211a corresponding to the icon A14 is selected, and the selection is provisionally determined. The pointer P1 moves when the distal end of the surgical tool unit 211A moves in accordance with an operation of the user Ul on the master robot 11.

In Step S104, when the pointer P1 is removed from above the icon A14 after the provisional determination, the provisionally determined selection is canceled. In contrast, in Step S105, when a certain time (e.g., five seconds) elapses with the pointer Pl being kept after the provisional determination, the provisionally determined selection is finally determined. In an example of FIG. 11, an image G11 indicating the elapse of the certain time is displayed.

After the final determination, the slave robot 21 can shift to the autonomous motion task. That is, the distal end of the surgical tool unit 211A is located in the interference avoidance region R100 by locating the distal end of the surgical tool unit 211A that moves the pointer P1 in a predetermined area of the surgical tool selection image G10. Furthermore, the distal end of the surgical tool unit 211B is also located in the interference avoidance region R100 in a similar flow. This permits execution of the surgical tool replacement task, which is an autonomous motion task of the slave robot 21. Thereafter, work of replacing the surgical tool 211a of the slave robot 21 with the finally determined surgical tool 211a is automatically executed.

In such processing, for example, the display control unit 312 provides the surgical tool selection image G10 for guiding the distal end of the slave robot 21 into the interference avoidance region R100 at the time of shift of the slave robot 21 to the autonomous motion task. This eliminates the need for calculation related to interference avoidance at the time of shift of the slave robot 21 to the autonomous motion task. Specifically, the surgical tool selection image G10 is displayed on the screen of the display unit 13. The master robot 11 is operated to use the distal end of the slave robot 21 as an operation device. In the case, the distal end of the slave robot 21 can be located in the interference avoidance region R100 by bringing the pointer Pl, that is, the distal end of the slave robot 21 to the predetermined area of the surgical tool selection image G10. Since the distal end of the slave robot 21 functions as the pointer Pl, the distal end of the slave robot 21 naturally comes to the predetermined area of the surgical tool selection image G10 at the time of selecting a surgical tool.

For example, in the master-slave system 1, a UI operation displayed on a screen is performed by moving the slave arm 212 with the master arm 111. At that time, a state in which the interference between the distal end of the slave robot 21 and an environment is avoided is preliminarily created by UI design. This eliminates the need for calculation for avoiding the interference between the distal end of the slave robot 21 and an environment in the subsequent motion of the slave robot 21. A prompt slave motion and prompt task start can thereby be achieved. Furthermore, the UI operation is performed by moving the slave arm 212 with the master arm 111, which eliminates the need for preliminarily recognizing an object with which interference is desired to be avoided.

### (Prediction of Replacement Surgical Tool)

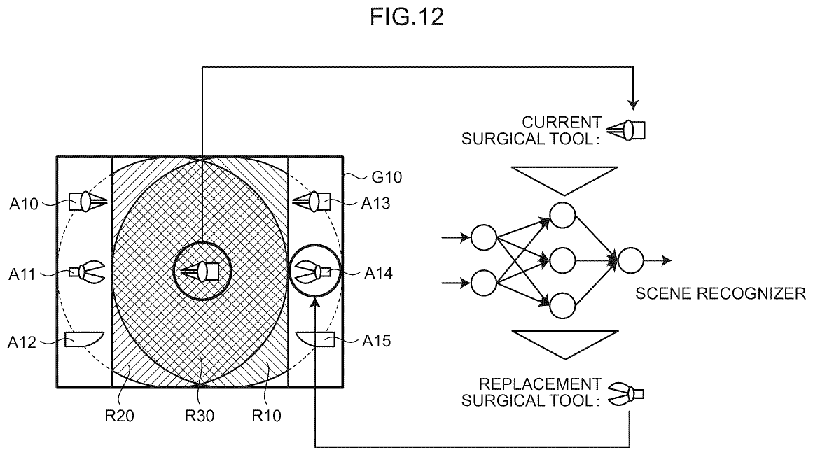

As illustrated in FIG. 12, the display control unit 312 recognizes the type of a current surgical tool used by the slave robot 21. Thereafter, the display control unit 312 predicts a replacement surgical tool replaced with which the recognized current surgical tool is to be replaced. The icon A14 corresponding to the predicted replacement surgical tool is displayed. In an example of FIG. 12, a scene recognizer of the display control unit 312 is used. For example, the scene recognizer recognizes the current surgical tool or predicts the replacement surgical tool by recognizing a surgical scene and a surgical tool through machine learning such as deep learning.

### (Automatic Movement of Distal End of Slave Robot 21)

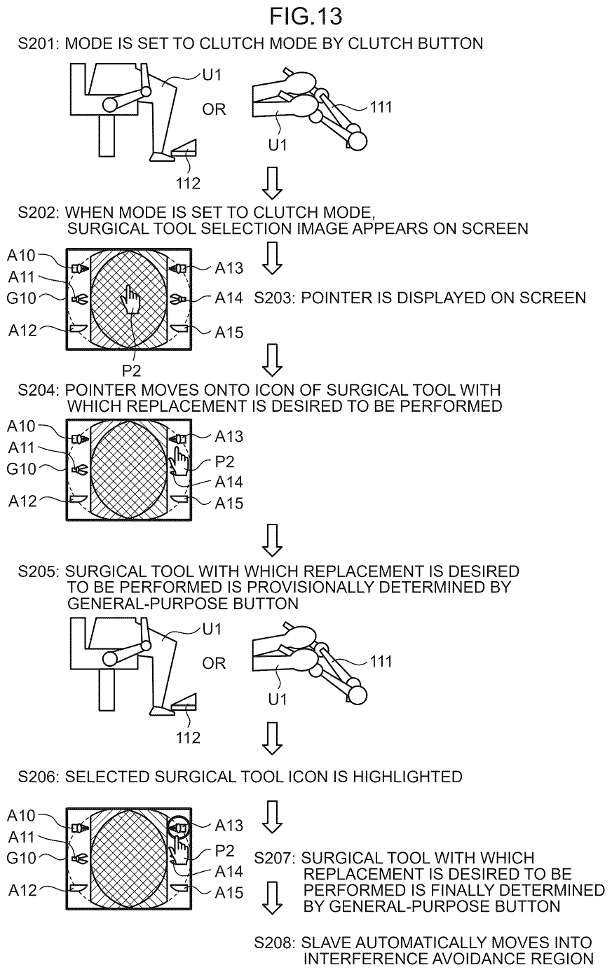

As illustrated in FIG. 13, in Step S201, a mode is set to a clutch mode by a clutch button (e.g., pressing clutch button). The user U1 presses the clutch button. Examples of the clutch button include the foot switch 112 and a switch provided on the master arm 111.

In Step S202, when the mode is set to the clutch mode, the surgical tool selection image G10 appears on the screen of the display unit 13. The surgical tool selection image G10 is used for generating a trigger for moving the distal end of the slave robot 21, that is, the individual distal ends of the surgical tool units 211A and 211B into the preset interference avoidance region R100. In Step S203, a pointer P2 is displayed on the screen. In Step S204, the pointer P2 moves onto an icon A13 of the surgical tool 211a with which replacement is desired to be performed. For example, the user U1 operates the master arm 111 to move the pointer P2. Unlike the pointer P1, the pointer P2 does not correspond to the distal end of the surgical tool unit 211A, but moves in accordance with the operation of the

In Step S205, the surgical tool 211a with which replacement is desired to be performed is provisionally determined by a general-purpose button (e.g., pressing general-purpose button). Examples of the general-purpose button include the foot switch 112 and a switch provided on the master arm 111. In Step S206, the selected surgical tool icon is highlighted. In the case, a confirmation dialog may pop up. In Step S207, the surgical tool 211a with which replacement is desired to be performed is finally determined by a general-purpose button (e.g., pressing general-purpose button).

In Step S208, the distal end of the slave robot 21 (individual distal ends of surgical tool units 211A and 211B of slave robot 21) automatically moves into the interference avoidance region R100. That is, the execution of the surgical tool replacement task, which is an autonomous motion task of the slave robot 21, is permitted by locating the individual distal ends of the surgical tool units 211A of the slave robot 21 in the interference avoidance region R100. Thereafter, work of replacing the surgical tool 211a of the slave robot 21 with the finally determined surgical tool 211a is automatically executed.

In such processing, for example, the display control unit 312 provides the surgical tool selection image G10 for generating a trigger for moving the distal end of the slave robot 21 into the interference avoidance region R100 at the time of shift of the slave robot 21 to the autonomous motion task. Thereafter, the distal end of the slave robot 21 automatically moves into the interference avoidance region R100. This eliminates the need for calculation related to interference avoidance at the time of shift of the slave robot 21 to the autonomous motion task. Therefore, a prompt slave motion and prompt task start can be achieved.

### 1-5. Processing Example of Movement of Distal End of Slave Robot Into Interference Avoidance Region

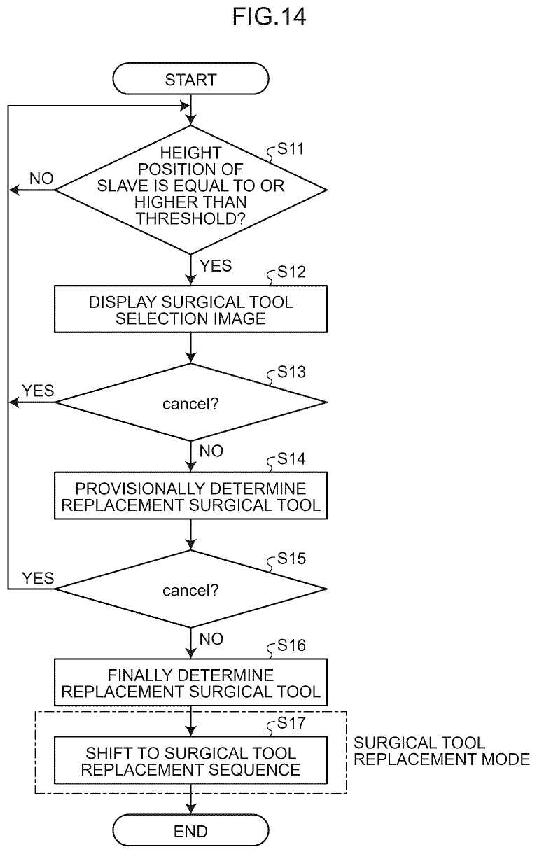

A processing example of the movement of the distal end of the slave robot 21 into the interference avoidance region R100 according to the embodiment will be described with reference to FIGS. 14 to 18. FIGS. 14 to 18 illustrate the processing example of the movement of the distal end of the slave robot 21 into the interference avoidance region R100 according to the embodiment.

### (First Example of Manual Movement of Distal End of Slave Robot 21)

As illustrated in FIG. 14, in Step S11, the display control unit 312 determines whether or not the height position of the distal end of the slave robot 21 is equal to or higher than a threshold. When determining that the height position of the distal end of the slave robot 21 is equal to or higher than the threshold (Yes in Step S11), the display control unit 312 causes the display unit 13 to display the surgical tool selection image G10 in Step S12. In contrast, when determining that the height position of the distal end of the slave robot 21 is not equal to or higher than the threshold (No in Step S11), the display control unit 312 returns the processing to Step S11.

In Step S13, the display control unit 312 determines whether or not there is cancellation in accordance with an operation of the user Ul on the master robot 11. When determining that there is no cancellation (No in Step S13), the display control unit 312 provisionally determines a replacement surgical tool in Step S14 in accordance with the operation of the user U1 on the master robot 11. In contrast, when determining that there is cancellation (Yes in Step S13), the display control unit 312 returns the processing to Step S11.

In Step S14 above, the distal end of the slave robot 21 functions as the pointer Pl (see FIGS. 10 and 11). The pointer P1 is superimposed and displayed on the surgical tool selection image G10. In response to the provisional determination of the replacement surgical tool, the pointer P1 moves onto a desired icon, for example, the icon A14 in a predetermined area of the surgical tool selection image G10. This moves the distal end of the slave robot 21 into the interference avoidance region R100. Furthermore, the provisionally determined icon A14 is highlighted, for example.

In Step S15, the display control unit 312 determines whether or not there is cancellation in accordance with an operation of the user U1 on the master robot 11. When determining that there is no cancellation (No in Step S15), the display control unit 312 finally determines a replacement surgical tool in Step S16 in accordance with the operation of the user U1 on the master robot 11. In contrast, when determining that there is cancellation (Yes in Step S15), the display control unit 312 returns the processing to Step S11.

In Step S17, the display control unit 312 shifts the processing to a surgical tool replacement sequence in order to replace the current surgical tool with the finally determined replacement surgical tool. Note that a motion mode is switched to a surgical tool replacement mode. The surgical tool replacement sequence will be described later in detail.

### (First Example of Automatic Movement of Distal End of Slave Robot 21)

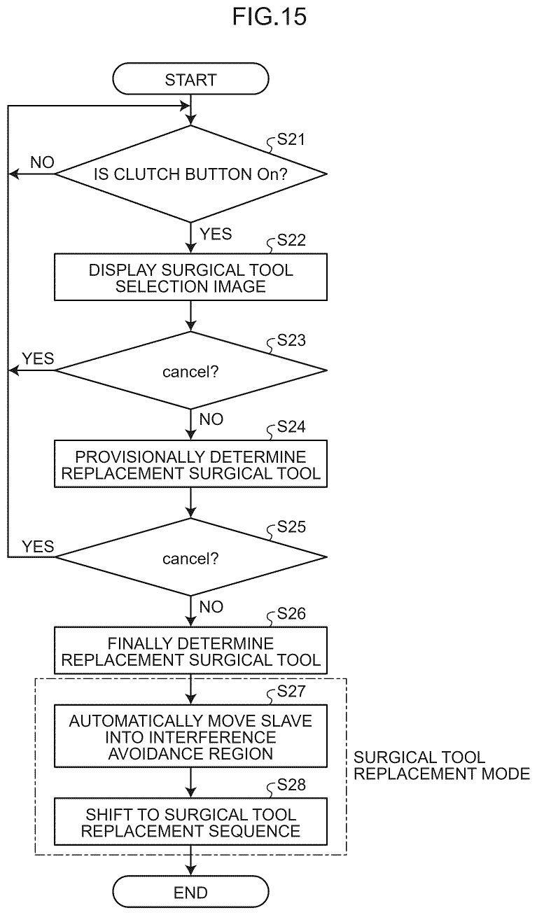

As illustrated in FIG. 15, in Step S21, the display control unit 312 determines whether or not the clutch button is On in accordance with an operation of the user Ul on the master robot 11. When determining that the clutch button is On (Yes in Step S21), the display control unit 312 displays the surgical tool selection image G10 on the display unit 13 in Step S22. In contrast, when determining that the clutch button is not On (No in Step S21), the display control unit 312 returns the processing to Step S21.

In Step S23, the display control unit 312 determines whether or not there is cancellation in accordance with an operation of the user U1 on the master robot 11. When determining that there is no cancellation (No in Step S23), the display control unit 312 provisionally determines a replacement surgical tool in Step S24 in accordance with the operation of the user U1 on the master robot 11. In contrast, when determining that there is cancellation (Yes in Step S23), the display control unit 312 returns the processing to Step S21.

In Step S24 above, the pointer P2 (see FIG. 13) is superimposed and displayed on the surgical tool selection image G10. In response to the provisional determination of the replacement surgical tool, the pointer P2 moves onto a desired icon, for example, the icon A13 in a predetermined area of the surgical tool selection image G10. Furthermore, the provisionally determined icon A13 is highlighted, for example.

In Step S25, the display control unit 312 determines whether or not there is cancellation in accordance with an operation of the user Ul on the master robot 11. When determining that there is no cancellation (No in Step S25), the display control unit 312 finally determines a replacement surgical tool in Step S26 in accordance with the operation of the user U1 on the master robot 11. In contrast, when determining that there is cancellation (Yes in Step S25), the display control unit 312 returns the processing to Step S21.

In Step S27, the drive control unit 311 automatically moves the distal end of the slave robot 21 into the interference avoidance region R100. In Step S28, the display control unit 312 shifts the processing to the surgical tool replacement sequence in order to replace the current surgical tool with the finally determined replacement surgical tool. Note that a motion mode is switched to a surgical tool replacement mode. The surgical tool replacement sequence will be described later in detail.

### (Second Example of Automatic Movement of Distal End of Slave Robot 21)

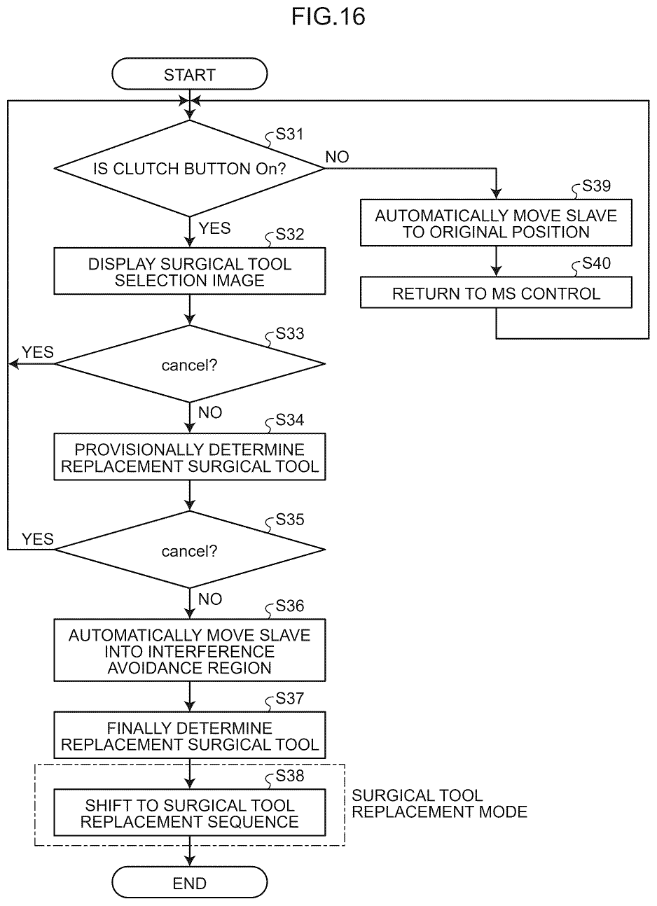

As illustrated in FIG. 16, in Step S31, the display control unit 312 determines whether or not the clutch button is On in accordance with an operation of the user Ul on the master robot 11. When determining that the clutch button is On (Yes in Step S31), the display control unit 312 displays the surgical tool selection image G10 on the display unit 13 in Step S32.

In Step S33, the display control unit 312 determines whether or not there is cancellation in accordance with an operation of the user Ul on the master robot 11. When determining that there is no cancellation (No in Step S33), the display control unit 312 provisionally determines a replacement surgical tool in Step S34 in accordance with the operation of the user U1 on the master robot 11. In contrast, when determining that there is cancellation (Yes in Step S33), the display control unit 312 returns the processing to Step S31.

In Step S34 above, the pointer P2 (see FIG. 13) is superimposed and displayed on the surgical tool selection image G10. In response to the provisional determination of the replacement surgical tool, the pointer P2 moves onto a desired icon, for example, the icon A13 in a predetermined area of the surgical tool selection image G10. Furthermore, the provisionally determined icon A13 is highlighted, for example.

In Step S35, the display control unit 312 determines whether or not there is cancellation in accordance with an operation of the user U1 on the master robot 11. When the display control unit 312 determines that there is no cancellation (No in Step S35), the drive control unit 311 automatically moves the distal end of the slave robot 21 into the interference avoidance region R100 in Step S36. In Step S37, the display control unit 312 finally determines the replacement surgical tool in accordance with an operation of the user U1 on the master robot 11. In contrast, when determining that there is cancellation (Yes in Step S35), the display control unit 312 returns the processing to Step S31.

In Step S38, the display control unit 312 shifts the processing to the surgical tool replacement sequence in order to replace the current surgical tool with the finally determined replacement surgical tool. Note that a motion mode is switched to a surgical tool replacement mode. The surgical tool replacement sequence will be described later in detail.

In contrast, when determining that the clutch button is not On in Step S31 above (No in Step S31), the display control unit 312 automatically moves the distal end of the slave robot 21 to the original position in Step S39, returns automatic control to master-slave control (MS control) in Step S40, and returns the processing to Step S31.

### (Third Example of Automatic Movement of Distal End of Slave Robot 21)

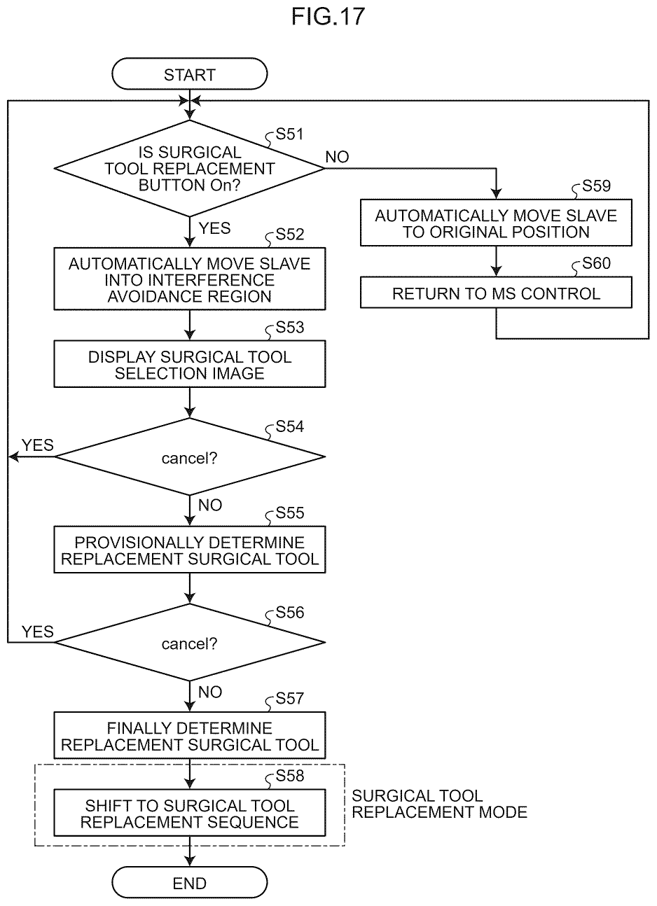

As illustrated in FIG. 17, in Step S51, the display control unit 312 determines whether or not a surgical tool replacement button is On in accordance with an operation of the user Ul on the master robot 11. When the display control unit 312 determines that the surgical tool replacement button is On (Yes in Step S51), the drive control unit 311 automatically moves the distal end of the slave robot 21 into the interference avoidance region R100 in Step S52. In Step S53, the display control unit 312 causes the display unit 13 to display the surgical tool selection image G10. Note that examples of the surgical tool replacement button include the foot switch 112 and a switch provided on the master arm 111.

In Step S54, the display control unit 312 determines whether or not there is cancellation in accordance with an operation of the user Ul on the master robot 11. When determining that there is no cancellation (No in Step S54), the display control unit 312 provisionally determines a replacement surgical tool in Step S55 in accordance with the operation of the user U1 on the master robot 11. In contrast, when determining that there is cancellation (Yes in Step S54), the display control unit 312 returns the processing to Step S51.

In Step S54 above, the pointer P2 (see FIG. 13) is superimposed and displayed on the surgical tool selection image G10. In response to the provisional determination of the replacement surgical tool, the pointer P2 moves onto a desired icon, for example, the icon A13 in a predetermined area of the surgical tool selection image G10. Furthermore, the provisionally determined icon A13 is highlighted, for example.

In Step S56, the display control unit 312 determines whether or not there is cancellation in accordance with an operation of the user Ul on the master robot 11. When determining that there is no cancellation (No in Step S56), the display control unit 312 finally determines a replacement surgical tool in Step S57 in accordance with the operation of the user U1 on the master robot 11. In contrast, when determining that there is cancellation (Yes in Step S56), the display control unit 312 returns the processing to Step S51.

In Step S58, the display control unit 312 shifts the processing to the surgical tool replacement sequence in order to replace the current surgical tool with the finally determined replacement surgical tool. Note that a motion mode is switched to a surgical tool replacement mode. The surgical tool replacement sequence will be described later in detail.

In contrast, when determining that the surgical tool replacement button is not On in Step S51 above (No in Step S51), the display control unit 312 automatically moves the distal end of the slave robot 21 to the original position in Step S59, returns automatic control to master-slave control in Step S60, and returns the processing to Step S51.

### (Fourth Example of Automatic Movement of Distal End of Slave Robot 21)

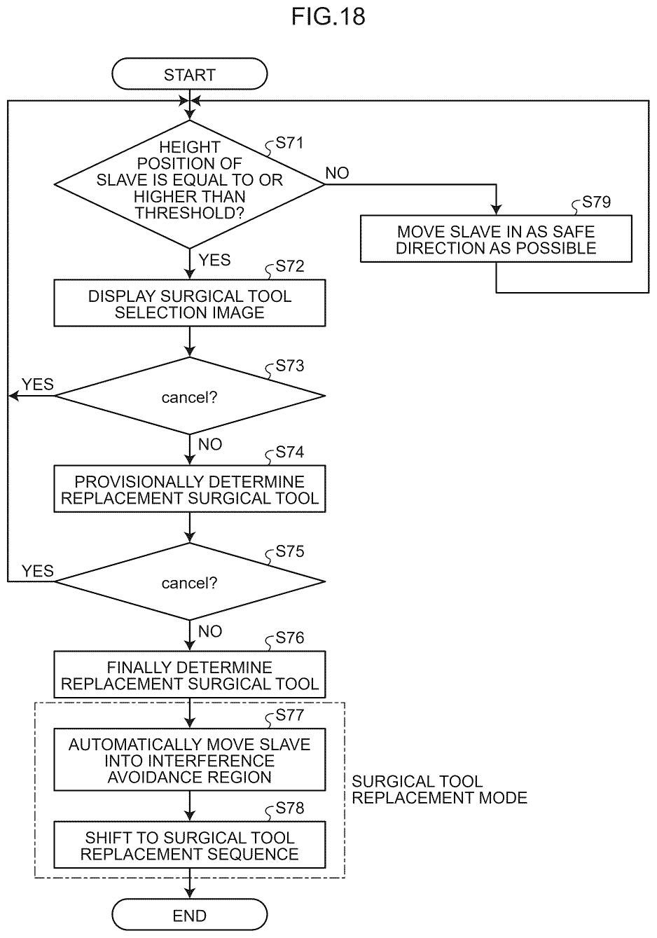

As illustrated in FIG. 18, in Step S71, the display control unit 312 determines whether or not the height position of the distal end of the slave robot 21 is equal to or higher than a threshold. When determining that the height position of the distal end of the slave robot 21 is equal to or higher than the threshold (Yes in Step S71), the display control unit 312 causes the display unit 13 to display the surgical tool selection image G10 in Step S72.

In Step S73, the display control unit 312 determines whether or not there is cancellation in accordance with an operation of the user U1 on the master robot 11. When determining that there is no cancellation (No in Step S73), the display control unit 312 provisionally determines a replacement surgical tool in Step S74 in accordance with the operation of the user U1 on the master robot 11. In contrast, when determining that there is cancellation (Yes in Step S73), the display control unit 312 returns the processing to Step S71.

In Step S74 above, the pointer P2 (see FIG. 13) is superimposed and displayed on the surgical tool selection image G10. In response to the provisional determination of the replacement surgical tool, the pointer P2 moves onto a desired icon, for example, the icon A13 in a predetermined area of the surgical tool selection image G10. Furthermore, the provisionally determined icon A13 is highlighted.

In Step S75, the display control unit 312 determines whether or not there is cancellation in accordance with an operation of the user Ul on the master robot 11. When determining that there is no cancellation (No in Step S75), the display control unit 312 finally determines a replacement surgical tool in Step S76 in accordance with the operation of the user U1 on the master robot 11. In contrast, when determining that there is cancellation (Yes in Step S75), the display control unit 312 returns the processing to Step S71.

In Step S77, the drive control unit 311 automatically moves the distal end of the slave robot 21 into the interference avoidance region R100. In Step S78, the display control unit 312 shifts the processing to the surgical tool replacement sequence in order to replace the current surgical tool with the finally determined replacement surgical tool. Note that a motion mode is switched to a surgical tool replacement mode. The surgical tool replacement sequence will be described later in detail.

In contrast, when the display control unit 312 determines that the height position of the distal end of the slave robot 21 is not equal to or higher than the threshold in Step S71 above (No in Step S71), the drive control unit 311 moves the distal end of the slave robot 21 in as safe a direction as possible in Step $79, and returns the processing to Step S71. The safe direction is a direction in which the distal end of the slave robot 21 moves toward the interference avoidance region R100.

### 1-6. Processing Example of Surgical Tool Replacement Sequence

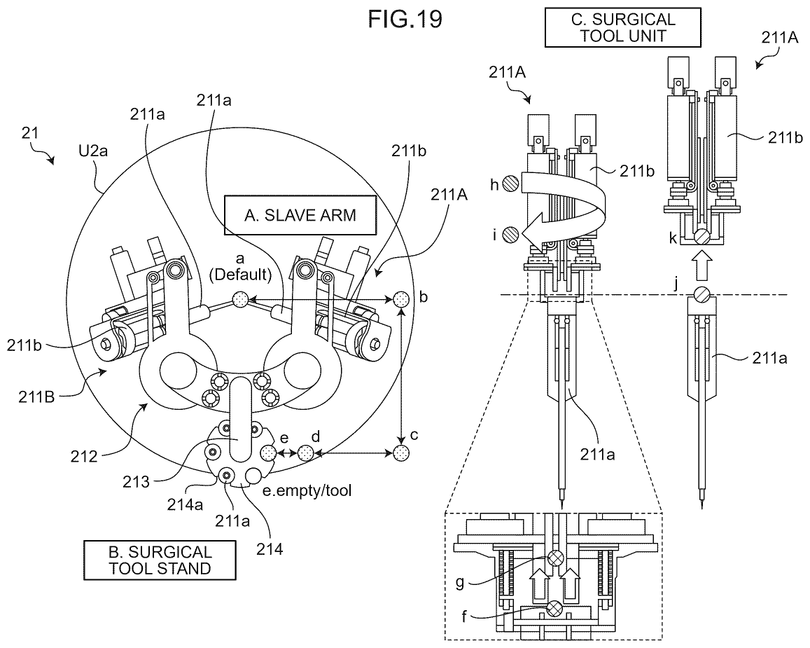

A processing example of the surgical tool replacement sequence according to the embodiment will be described with reference to FIGS. 19 to 32. FIGS. 19 to 32 illustrate the processing example of the surgical tool replacement sequence according to the embodiment.

### (First Processing Example)

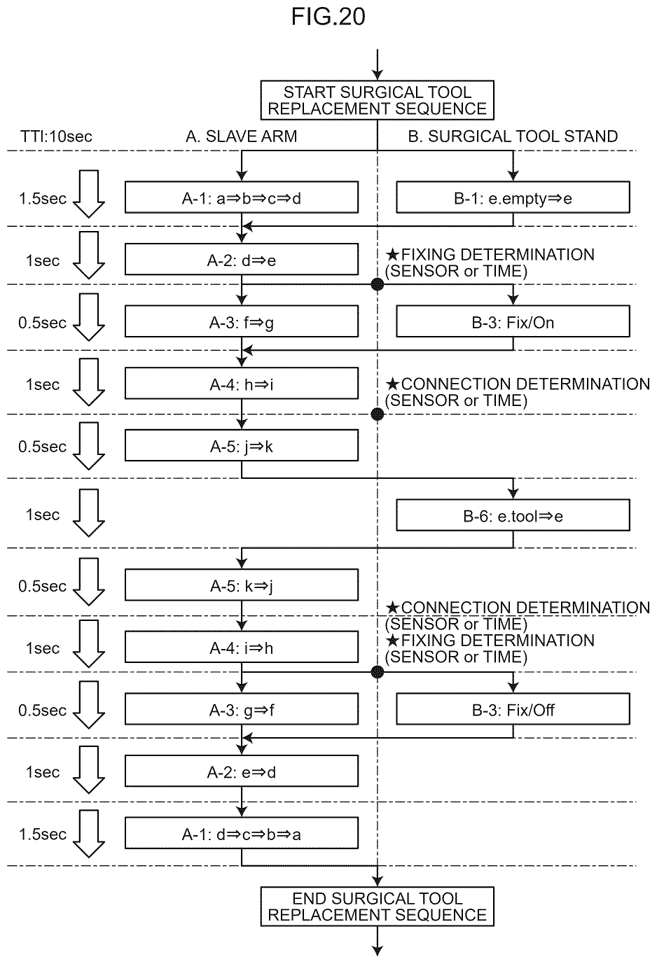

As illustrated in FIG. 19, for example, states (positions) a, b, c, d, e, f, g, h, i, j, and k are set. These states (positions) a to k correspond to a to k in FIG. 20. The surgical tool unit 211A of the slave arm 212 moves in a←→b←→c←→d←→e. Furthermore, the surgical tool 211a and the drive unit 211b of the surgical tool unit 211A of the slave arm 212 are disconnected in h→i (f→g), and separated in j→k. In contrast, the surgical tool 211a and the drive unit 211b are engaged in k→j, and connected in i→h (g→f). Note that the same applies to the surgical tool unit 211B except that the loci of the states (positions) a, b, c, d, and e are line-symmetric.

Note that, in order to recognize the surgical tool 211a stored in the surgical tool stand 214, the surgical tool 21la being used, and the like, an electronic tag such as a wireless tag may be provided in the surgical tool 211a, and the surgical tool 211a may be managed by the electronic tag. For example, the electronic tag includes a name and identification information of the surgical tool 211a. Furthermore, the external shape of the surgical tool 211a may be recognized and managed. Alternatively, the external shape of the surgical tool 211a may be mechanically managed. Furthermore, the surgical tool 211a may be fixed and connected by, for example, a fixing structure and a connection structure.

FIG. 20 illustrates motion orders of A: slave arm (slave arm 212) and B: surgical tool stand (surgical tool stand 214). Fixing determination or connection determination is made based on a detection result from the sensor unit 25 or the elapse of a predetermined time. The fixing determination or the connection determination is appropriately made, for example. Note that, in an example of FIG. 20, the start point is set as a. For example, however, when the distal end of the surgical tool unit 211A is located in the interference avoidance region R100, the start point is set as not a but b.

In the first 1.5 sec, in A-1, the surgical tool unit 211A of the slave arm 212 moves in a→b→c→d (A-1: a→b→c→d). In B-1, the surgical tool stand 214 rotates to a position where the empty storage portion 214a faces the surgical tool unit 211A of the slave arm 212 (B-1: e.empty→e).

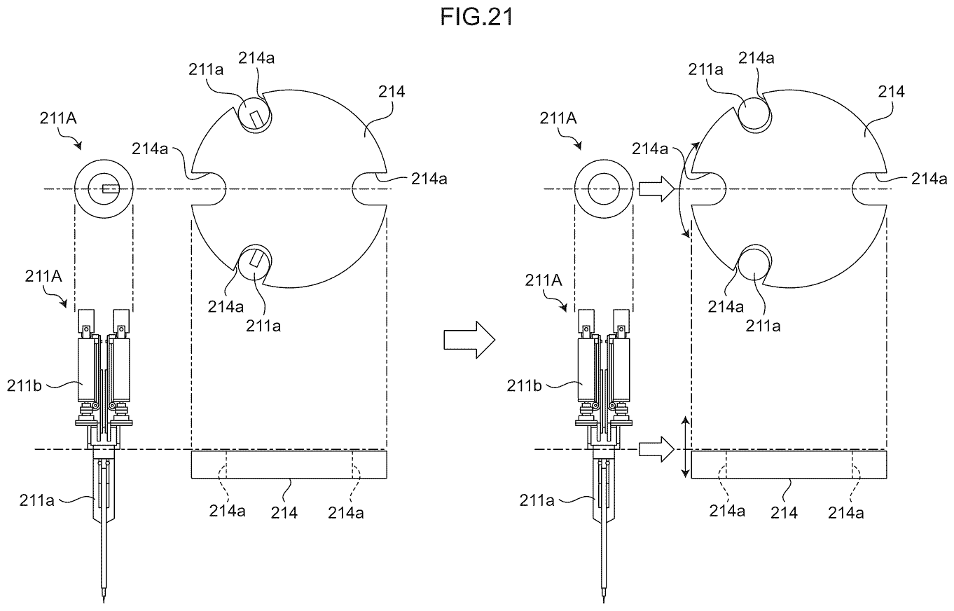

In the next 1 sec, in A-2, the surgical tool unit 211A of the slave arm 212 moves in d→e (A-2: d→e). Here, the fixing determination is made. As illustrated in FIG. 21, when the surgical tool unit 211A of the slave arm 212 is located at d, a movement is made toward a position in the empty storage portion 214a.

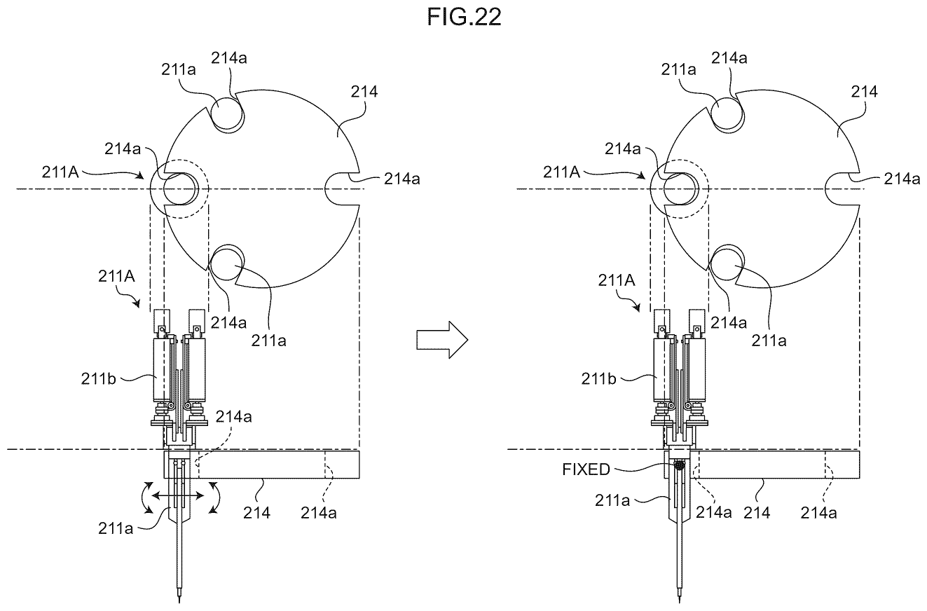

Returning to FIG. 20, in the next 0.5 sec, in A-3, the surgical tool unit 211A of the slave arm 212 moves in f→g (A-3: f→g). In B-3, the surgical tool unit 211A of the slave arm 212 is fixed after being aligned (B-3: Fix/On). As illustrated in FIG. 22, the surgical tool unit 211A of the slave arm 212 is aligned when located in the empty storage portion 214a, and the surgical tool 211a of the surgical tool unit 211A is fixed.

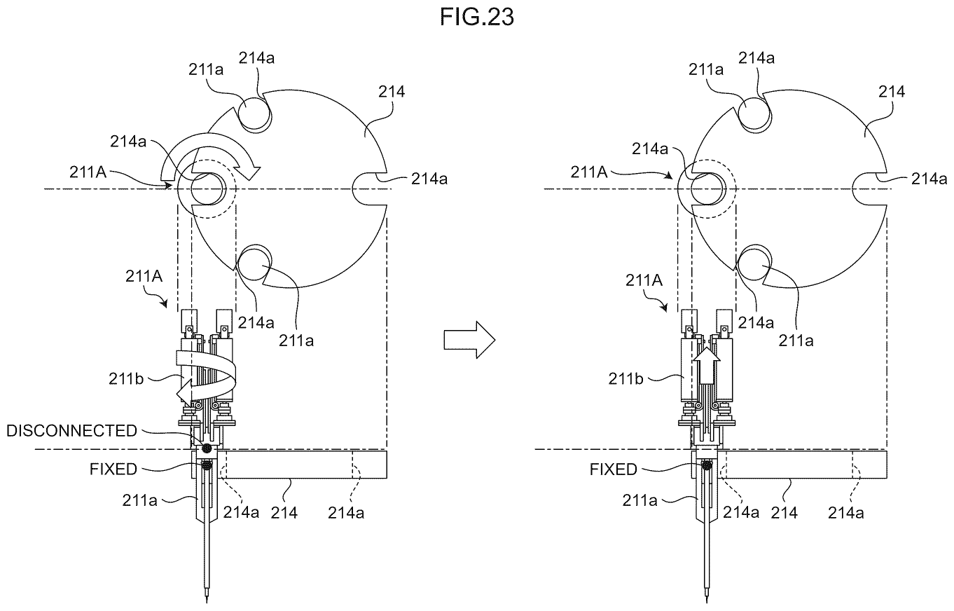

Returning to FIG. 20, in the next 1 sec, in A-4, the surgical tool unit 211A of the slave arm 212 rotates in h→i (A-4: h→i). Here, the connection determination is made. As illustrated in FIG. 23 (see left figure in FIG. 23), the drive unit 211b of the surgical tool unit 211A of the slave arm 212 rotates. The surgical tool unit 211A is disconnected from the surgical tool 211a. Note that the surgical tool 211a remains fixed.

Returning to FIG. 20, in the next 0.5 sec, in A-5, the surgical tool unit 211A of the slave arm 212 is disassembled. The drive unit 211b moves in j→k while the surgical tool 211a remains fixed (A-5: j→k). As illustrated in FIG. 23 (see right figure in FIG. 23), the drive unit 211b of the surgical tool unit 211A of the slave arm 212 moves upward after the surgical tool unit 211A is disconnected from the surgical tool 211a.

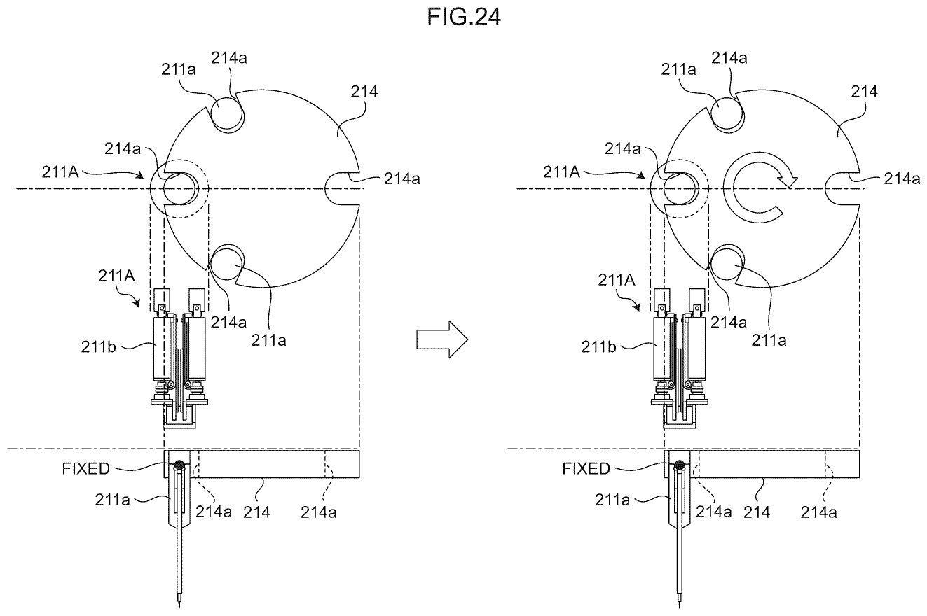

Returning to FIG. 20, in the next 1 sec, in B-6, the surgical tool stand 214 rotates until the surgical tool 211a with which replacement is desired to be performed is located immediately below the drive unit 211b (B-6: e. tool→e). As illustrated in FIG. 24, when the drive unit 211b of the surgical tool unit 211A of the slave arm 212 move upward and is separated from the drive unit 211b, the surgical tool stand 214 rotates until the surgical tool 211a with which replacement is desired to be performed is located immediately below the drive unit 211b.

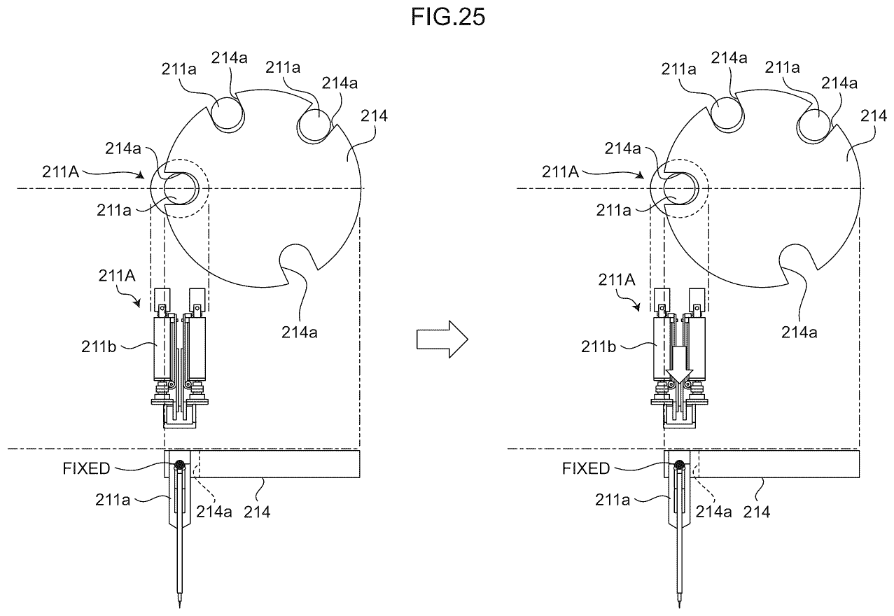

Returning to FIG. 20, in the next 0.5 sec, in A-5, the drive unit 211b moves in k→j while the surgical tool 211a of the surgical tool unit 211A of the slave arm 212 remains fixed (A-5: k→j). As illustrated in FIG. 25, the drive unit 211b of the surgical tool unit 211A of the slave arm 212 moves downward when the surgical tool 211a with which replacement is desired to be performed is located immediately below the drive unit 211b.

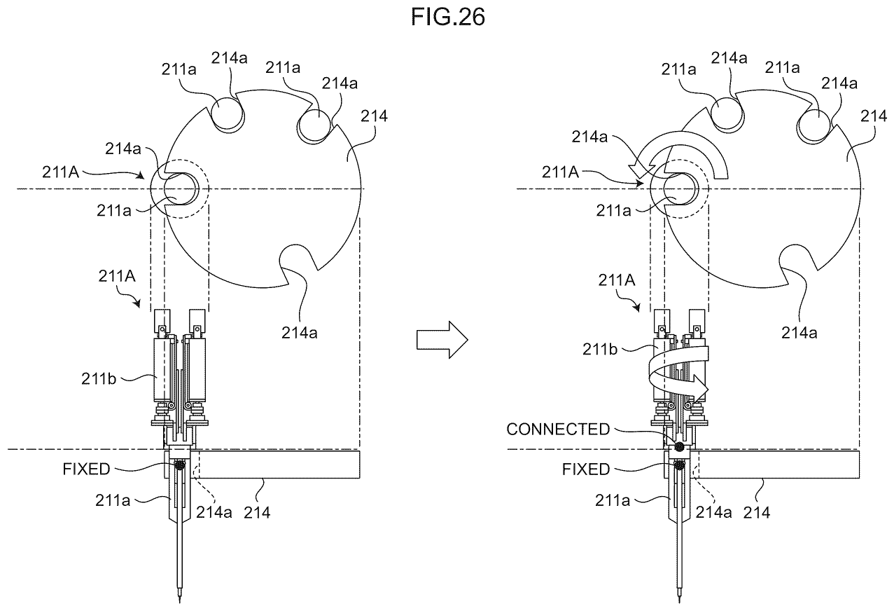

Returning to FIG. 20, in the next 1 sec, in A-4, the drive unit 211b of the surgical tool unit 211A of the slave arm 212 rotates in i→h (A-4: i→h). Here, the connection determination and the fixing determination are made. As illustrated in FIG. 26, the drive unit 211b of the surgical tool unit 211A of the slave arm 212 rotates when being engaged with the surgical tool 211a with which replacement is desired to be performed, and is connected to the surgical tool 211a with which replacement is desired to be performed.

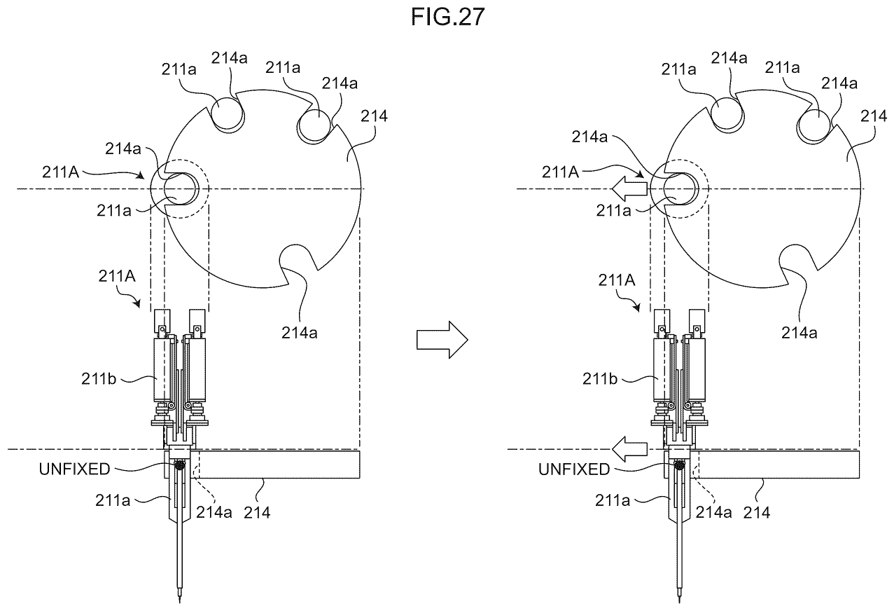

Returning to FIG. 20, in the next 0.5 sec, in A-3, the surgical tool unit 211A of the slave arm 212 moves in g->f (A-3: g→f). In B-3, the surgical tool unit 211A of the slave arm 212 is unfixed (B-3: Fix/Off). As illustrated in FIG. 27 (see left figure in FIG. 27), the surgical tool 211a of the surgical tool unit 211A of the slave arm 212 is unfixed.

Returning to FIG. 20, in the next 1 sec, in A-2, the surgical tool unit 211A of the slave arm 212 moves in e→d (A-2: e→d). As illustrated in FIG. 27 (see right figure in FIG. 27), when the surgical tool 211a of the surgical tool unit 211A of the slave arm 212 is unfixed, the surgical tool unit 211A of the slave arm 212 moves.

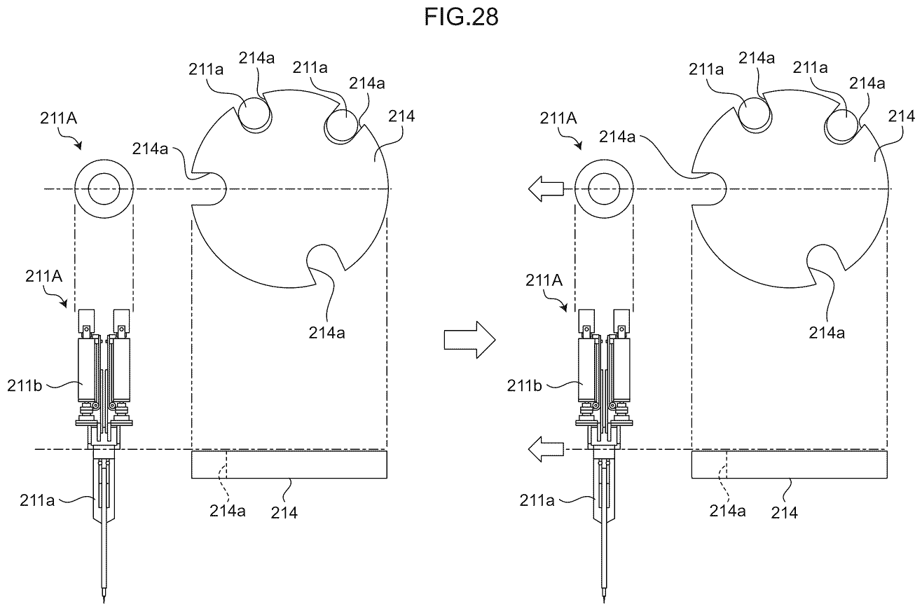

Returning to FIG. 20, in the last 1.5 sec, in A-1, the surgical tool unit 211A of the slave arm 212 moves in d→c→b→a (A-1: d→c→b→a). As illustrated in FIG. 28, when located at d, the surgical tool unit 211A of the slave arm 212 moves in d→c→b→a.

### (Second Processing Example)

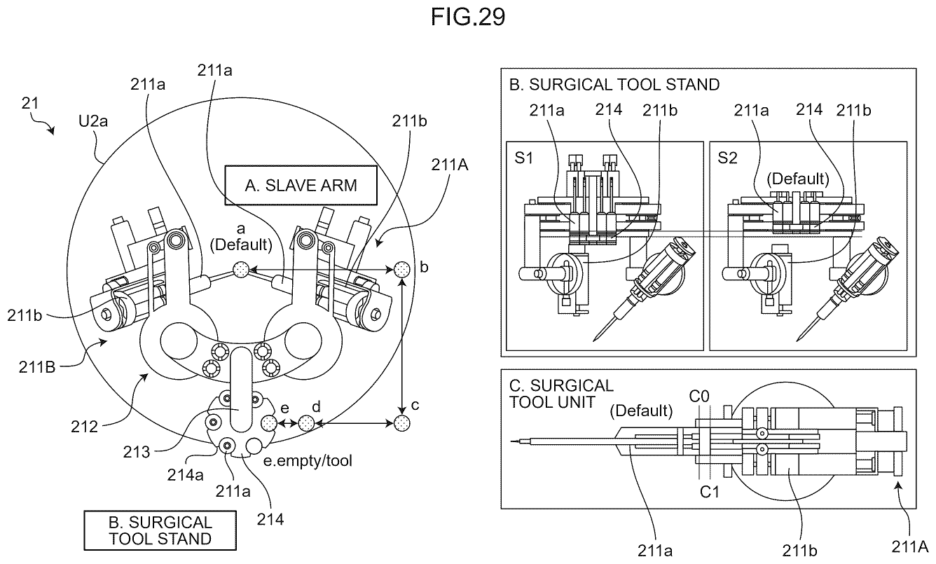

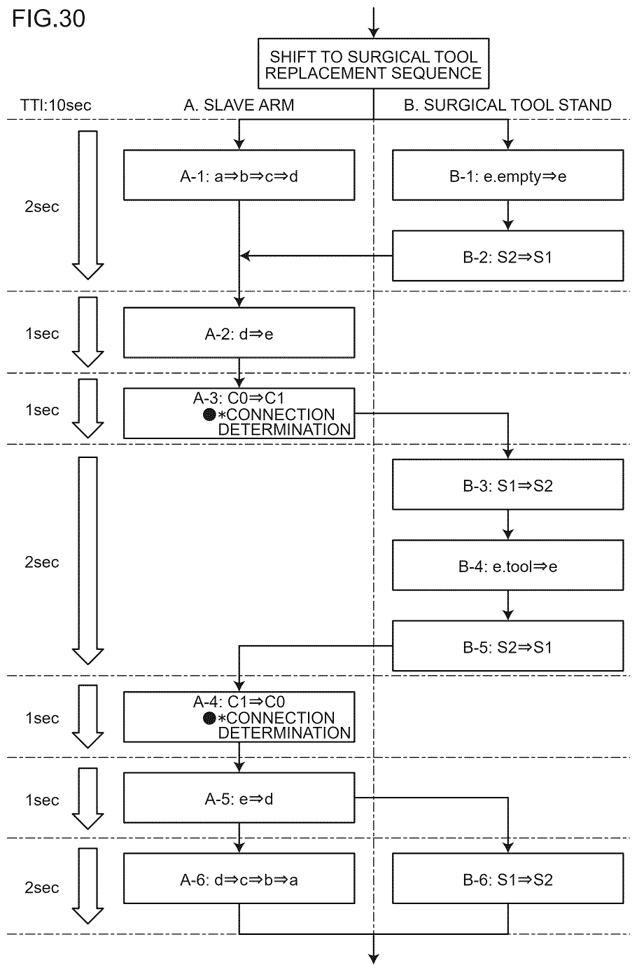

As illustrated in FIG. 29, for example, states (positions) a, b, c, d, e, S1, S2, C0, and C1 are set. The states (positions) a, b, c, d, e, S1, S2, C0, and C1 correspond to a, b, c, d, e, S1, S2, C0, and C1 in FIG. 30. The surgical tool unit 211A of the slave arm 212 moves in a←→b←→c←→d←→e. Furthermore, the surgical tool 211a and the drive unit 211b of the surgical tool unit 211A of the slave arm 212 are disconnected in C0→C1, and separated in S1→S2. In contrast, the surgical tool 211a and the drive unit 211b are engaged in S2→S1, and connected in C1→C0. Note that the same applies to the surgical tool unit 211B except that the loci of the states (positions) a, b, c, d, and e are line-symmetric.

FIG. 30 illustrates motion orders of A: slave arm (slave arm 212) and B: surgical tool stand (surgical tool stand 214). Connection determination is made based on a detection result from the sensor unit 25 or the elapse of a predetermined time. The connection determination is appropriately made, for example. Note that, in an example of FIG. 30, the start point is set as a. For example, however, when the distal end of the surgical tool unit 211A is located in the interference avoidance region R100, the start point is set as not a but b. Note that the surgical tool stand 214 can move in the height direction.

In the first 2 sec, in A-1, the surgical tool unit 211A of the slave arm 212 moves in a→b→c→d (A-1: a→b→c→d). In B-1, the surgical tool stand 214 rotates to a position where the empty storage portion 214a faces the surgical tool unit 211A of the slave arm 212 (B-1: e.empty→e). In B-2, the surgical tool stand 214 moves in S2→S1 (B-2: S2→S1).

In the next 1 sec, in A-2, the surgical tool unit 211A of the slave arm 212 moves in d→e (A-2: d→e). In the next 1 sec, in A-3, the surgical tool unit 211A of the slave arm 212 moves in C0→C1 (A-3: C0→C1). Here, the connection determination is executed.

In the next 2 sec, in B-3, the surgical tool stand 214 moves in S1→S2 (B-3: S1→S2). In B-4, the surgical tool stand 214 rotates until the surgical tool 211a with which replacement is desired to be performed is located immediately below the drive unit 211b (B-4: e.tool→e). In B-5, the surgical tool stand 214 moves in S2→S1 (B-5: S2→S1).

In the next 1 sec, in A-4, the surgical tool unit 211A of the slave arm 212 moves in C1→C0 (A-4: C1→C0). Here, the connection determination is executed. In the next 1 sec, in A-5, the surgical tool unit 211A of the slave arm 212 moves in e→d (A-5: e→d).

In the last 2 sec, in A-6, the surgical tool unit 211A of the slave arm 212 moves in d→c→b→a (A-6: d→c→b→a). In B-5, the surgical tool stand 214 moves in S1→S2 (B-6: S1→S2).

### (Surgical Tool Attaching Mode)

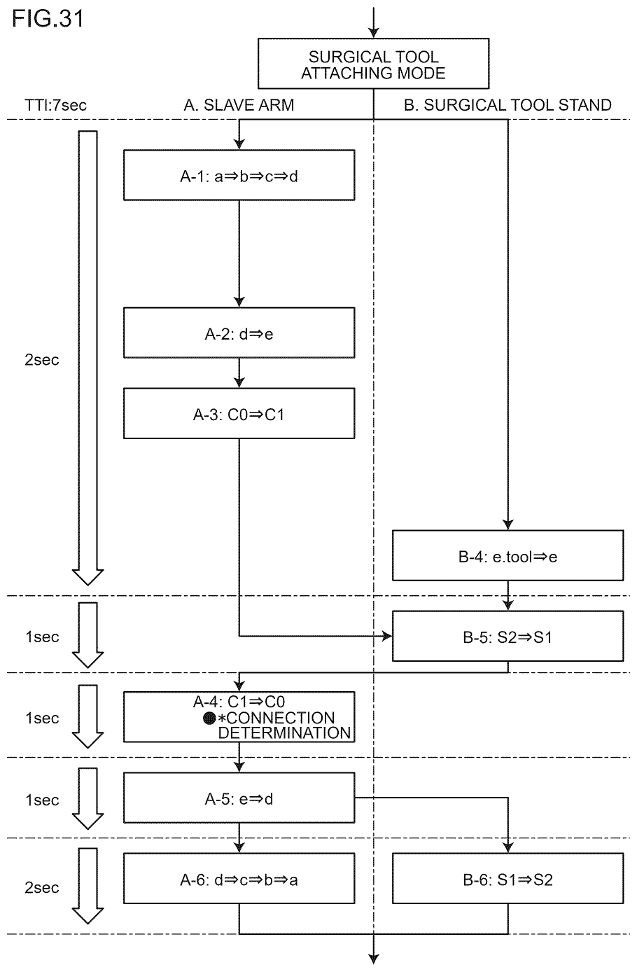

FIG. 31 illustrates motion orders of A: slave arm (slave arm 212) and B: surgical tool stand (surgical tool stand 214) in a surgical tool attaching mode.

In the first 2 sec, in A-1, the surgical tool unit 211A of the slave arm 212 moves in a→b→c→d (A-1: a→b→c→d). In A-2, the surgical tool unit 211A of the slave arm 212 moves in d→e (A-2: d→e). In A-3, the surgical tool unit 211A of the slave arm 212 moves in C0→C1 (A-3: C0→C1). In B-4, the surgical tool stand 214 rotates until the surgical tool 211a with which replacement is desired to be performed is located immediately below the drive unit 211b (B-4: e.tool→e).

In the next 1 sec, in B-5, the surgical tool stand 214 moves in S2→S1 (B-5: S2→S1). In the next 1 sec, in A-4, the surgical tool unit 211A of the slave arm 212 moves in C1→C0 (A-4: C1→C0). Here, the connection determination is executed. In the next 1 sec, in A-5, the surgical tool unit 211A of the slave arm 212 moves in e→d (A-5: e→d).

In the last 2 sec, in A-6, the surgical tool unit 211A of the slave arm 212 moves in d→c→b→a (A-6: d→c→b→a). In B-5, the surgical tool stand 214 moves in S1→S2 (B-6: S1→S2).

### (Surgical Tool Detaching Mode)

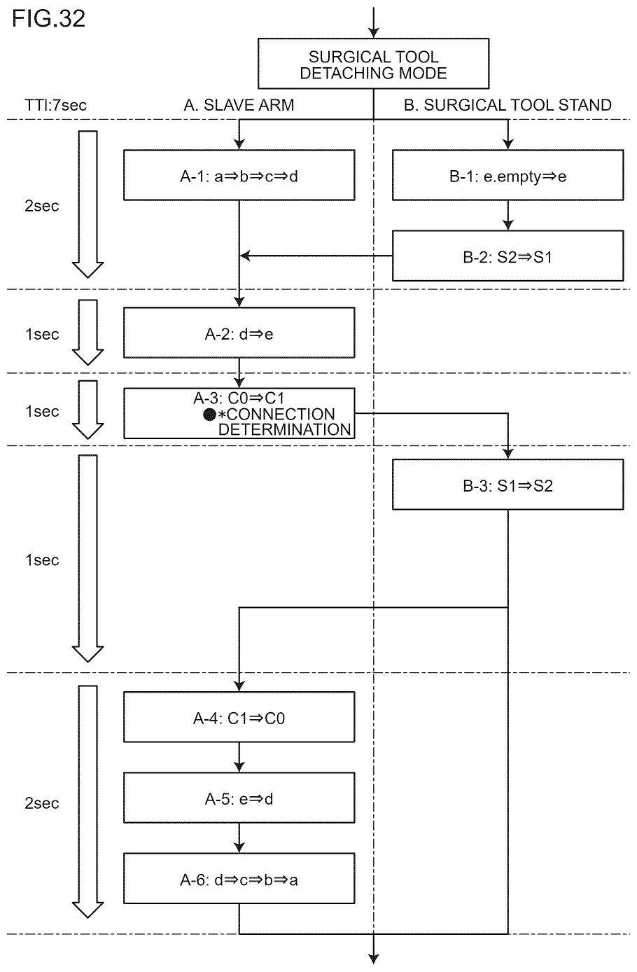

FIG. 32 illustrates motion orders of A: slave arm (slave arm 212) and B: surgical tool stand (surgical tool stand 214) in a surgical tool detaching mode.

In the first 2 sec, in A-1, the surgical tool unit 211A of the slave arm 212 moves in a→b→c→d (A-1: a→b→c→d). In B-1, the surgical tool stand 214 rotates to a position where the empty storage portion 214a faces the surgical tool unit 211A of the slave arm 212 (B-1: e.empty→e). In B-2, the surgical tool stand 214 moves in S2→S1 (B-2: S2→S1).

In the next 1 sec, in A-2, the surgical tool unit 211A of the slave arm 212 moves in d→e (A-2: d→e). In the next 1 sec, in A-3, the surgical tool unit 211A of the slave arm 212 moves in C0→C1 (A-3: C0→C1). Here, the connection determination is executed. In the next 1 sec, in B-3, the surgical tool stand 214 moves in S1→S2 (B-3: S1→S2).

In the last 2 sec, in A-4, the surgical tool unit 211A of the slave arm 212 moves in C1→CO (A-4: C1→C0). In A-5, the surgical tool unit 211A of the slave arm 212 moves in e→d (A-5: e→d). In A-6, the surgical tool unit 211A of the slave arm 212 moves in d→c→b→a (A-6: d→c→b→a).

### 1-7. Effects

As described above, according to the embodiment, the master-slave system 1 includes the display unit 13 and the display control unit 312. The display unit 13 displays an image to the user U1 who operates the master robot 11. The display control unit 312 causes the display unit 13 to display an image (e.g., surgical tool selection image G10) for locating the distal end of the slave robot 21 in the preset interference avoidance region R100. This enables the distal end of the slave robot 21 to be located in the interference avoidance region R100, and eliminates the need for calculation related to interference avoidance, for example. Therefore, a prompt slave motion and prompt task start can be achieved.

Furthermore, the image may be used for guiding the distal end of the slave robot 21 into the interference avoidance region R100. This enables the distal end of the slave robot 21 to be reliably located in the interference avoidance region R100.

Furthermore, the image may be used for generating a trigger for moving the distal end of the slave robot 21 into the interference avoidance region R100. This enables the distal end of the slave robot 21 to be reliably located in the interference avoidance region R100.

Furthermore, the image may be a tool selection image (e.g., surgical tool selection image G10) including an icon representing a tool (e.g., surgical tool 211a). The icon may be arranged at a position corresponding to the interference avoidance region R100 in the tool selection image. This enables the distal end of the slave robot 21 to be reliably located in the interference avoidance region R100.

Furthermore, the tool selection image may include a plurality of icons. The plurality of icons may be arranged at positions corresponding to the interference avoidance region R100 in the tool selection image. This enables the distal end of the slave robot 21 to be reliably located in the interference avoidance region R100.

Furthermore, the display control unit 312 may predict a tool with which replacement is to be performed from a tool of the slave robot 21 and a scene, and display an icon representing the predicted tool. This enables an appropriate tool to be presented to the user U1.

Furthermore, the tool may be the surgical tool 211a. The tool selection image may be the surgical tool selection image G10. This enables the distal end of the slave robot 21 to be reliably located in the interference avoidance region R100 in the medical field.

Furthermore, the interference avoidance region R100 may be a region where the height position of the distal end of the slave robot 21 is equal to or higher than a predetermined threshold. This can avoid the interference between the distal end of the slave robot 21 and the surrounding environment (e. g., user U2 and bed U2a) of the slave robot 21.

Furthermore, the predetermined threshold may be set based on a predetermined separation distance between the distal end of the slave robot 21 and the user U2, who is a patient. This can avoid the interference between the distal end of the slave robot 21 and the user U2.

Furthermore, the slave robot 21 may include a plurality of distal ends (e.g., individual distal ends of surgical tool units 211A and 211B). The interference avoidance region R100 may be a region excluding the movable region R30 common to the plurality of distal ends of the slave robot 21. This can avoid the interference between the distal ends of the slave robot 21.

Furthermore, the interference avoidance region R100 may include the movable regions R10 and R20 for each of the distal ends of the slave robot 21. This enables the distal end of the slave robot 21 to be reliably located in the interference avoidance region R100.

Furthermore, when the height position of the distal end of the slave robot 21 is equal to or higher than a predetermined threshold, the display control unit 312 may cause the display unit 13 to display an image. This enables the display unit 13 to display an image at appropriate timing.

Furthermore, the display control unit 312 may cause the display unit 13 to display an image in accordance with an operation of the user Ul on the master robot 11. This enables the display unit 13 to display an image at appropriate timing.

Furthermore, the moving speed of the distal end of the slave robot 21 in a case where the height position of the distal end of the slave robot 21 is equal to or higher than a predetermined height position may be set to be higher than the moving speed of the distal end of the slave robot 21 in a case where the height position of the distal end of the slave robot 21 is lower than the predetermined height position. This can achieve a prompt slave motion.

Furthermore, the master-slave system 1 may further include the region setting unit 313 that sets the interference avoidance region R100. This can set the appropriate interference avoidance region R100.

Furthermore, the region setting unit 313 may set the interference avoidance region R100 such that the interference avoidance region R100 is a region where the height position of the distal end of the slave robot 21 is equal to or higher than a predetermined threshold. This can avoid the interference between the distal end of the slave robot 21 and the surrounding environment of the slave robot 21.

Furthermore, the master-slave system 1 may further include the drive control unit 311 that controls the slave robot 21 so that the distal end of the slave robot 21 moves into the interference avoidance region R100. This enables the distal end of the slave robot 21 to be reliably located in the interference avoidance region R100.

Furthermore, when the distal end of the slave robot 21 is located in the interference avoidance region R100, the drive control unit 311 may cause the slave robot 21 to execute a tool replacement operation for replacing a tool of the slave robot 21 with another tool. This enables the slave robot 21 to automatically execute the tool replacement operation when the distal end of the slave robot 21 is located in the interference avoidance region R100.

### 2. Other Embodiments

The configurations and the pieces of processing according to the above-described embodiment (examples and variations) may be performed in various different forms other than the above-described embodiment. For example, the configurations and the pieces of processing are not limited to the above-described examples, and may be achieved in various modes. For example, among pieces of processing described in the above-described embodiment, all or part of the processing described as being performed automatically can be performed manually, or all or part of the processing described as being performed manually can be performed automatically by a known method. Furthermore, the configurations, the processing procedures, specific names, and information including various pieces of data and parameters in the above document and drawings can be optionally changed unless otherwise specified. For example, various pieces of information in each figure are not limited to the illustrated information.

Furthermore, the configurations and the pieces of processing according to the above-described embodiment (examples and variations) are not necessarily required to be physically configured as illustrated. That is, the specific form of distribution/integration of each device is not limited to the illustrated one, and all or part thereof can be configured in a functionally or physically distributed/integrated manner in any unit in accordance with various loads and use situations.

Furthermore, the configurations and the pieces of processing according to the above-described embodiment (examples and variations) may be appropriately combined. For example, at least a part of an embodiment may be appropriately combined with at least a part of another embodiment. Furthermore, the effects in the embodiment are merely examples and not limitations. Other effects may be exhibited.

Note that, although, in the above-described embodiment (or variations), the embodiment in which the above-described technique is applied to surgery in the medical field has been mainly described, the gist of the above-described technique is not limited thereto. The above-described technique can be applied, in a wide variety of fields, to, for example, a remotely operated robot that performs precise work in a manufacturing factory, a construction site, and difficult-to-work space such as the universe and an operation console device for remote operation.

### 3. Configuration Example of Hardware

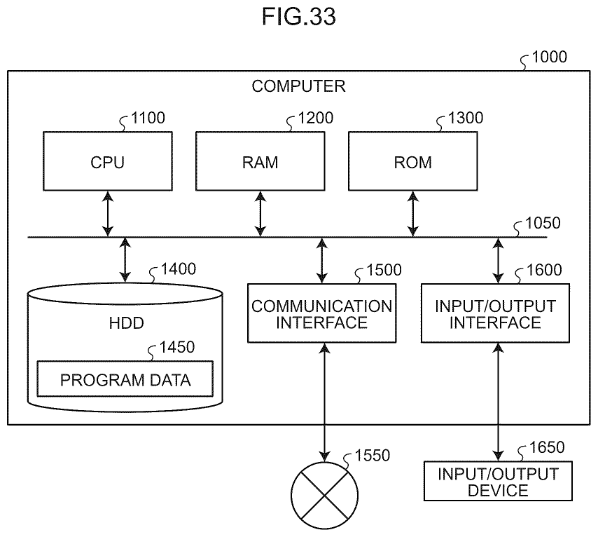

A specific hardware configuration example of various information devices according to the above embodiment (or modification) will be described. A part or all part, etc., of the various information devices (for example, the master device 10, the slave device 20, the control device 30) according to the embodiment (or the modification) may be realized by, for example, a computer 1000 having a configuration as illustrated in FIG. 33. FIG. 33 is a configuration example of hardware according to the embodiment.

As illustrated in FIG. 33, the computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates based on the program stored in the ROM 1300 or the HDD 1400, and controls each unit. For example, the CPU 1100 develops a program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a recording medium that can be read by the computer 1000 and performs non-transient recording of a program executed by the CPU 1100, data used by such a program, and the like. Specifically, the HDD 1400 is a recording medium that records a program for executing various processes according to the present disclosure, which is an example of program data 1450.

The communication interface 1500 is an interface for the computer 1000 to connect to an external network 580 (for example, the Internet). For example, the CPU 1100 receives data from another device or transmits data generated by the CPU 1100 to another device via the communication interface 1500.

The input/output interface 1600 is an interface for connecting an input/output device 1650 and the computer 1000. For example, the CPU 1100 receives data from an input device such as a keyboard or a mouse via the input/output interface 1600. In addition, the CPU 1100 transmits data to an output device such as a display, a speaker, or a printer via the input/output interface 1600.

Note that, in addition, the input/output interface 1600 may function as a media interface that reads a program and the like recorded in a predetermined recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, and the like.

Here, for example, in a case where the computer 1000 functions as the control device 30 and the like according to the embodiment, the CPU 1100 of the computer 1000 executes the program loaded on the RAM 1200 to realize the functions of the control device 30 and the like. In addition, the HDD 1400 stores programs and data for executing various processes according to the embodiment. Note that the CPU 1100 reads the program data 1450 from the HDD 1400 and executes the program data, but as another example, these programs may be acquired from another apparatus via the external network 1550.

### 4. Appendix

Note that the present technology can also have the configurations as follows.
(1) A master-slave system comprising:
   a display unit that displays an image to a user who operates a master robot; and
   a display control unit that causes the display unit to display an image for locating a distal end of a slave robot in an interference avoidance region that has been preset.
(2) The master-slave system according to (1),
   wherein the image is used for guiding the distal end of the slave robot into the interference avoidance region.
(3) The master-slave system according to (1),
   wherein the image is used for generating a trigger for moving the distal end of the slave robot into the interference avoidance region.
(4) The master-slave system according to any one of (1) to (3),
   wherein the image is a tool selection image including an icon representing a tool, and
   the icon is arranged at a position corresponding to the interference avoidance region in the tool selection image.
(5) The master-slave system according to (4),
   wherein the tool selection image includes a plurality of icons, and
   the plurality of icons is arranged at positions corresponding to the interference avoidance region in the tool selection image.
(6) The master-slave system according to (4) or (5),
   wherein the display control unit predicts a tool with which replacement is to be performed from a tool of the slave robot and a scene, and displays an icon representing the tool that has been predicted.
(7) The master-slave system according to any one of (4) to (6),
   wherein the tool is a surgical, and
   the tool selection image is a surgical tool selection image.
(8) The master-slave system according to any one of (1) to (7),
   the interference avoidance region is a region where a height position of the distal end of the slave robot is equal to or higher than a predetermined threshold.
(9) The master-slave system according to (8),
   wherein the predetermined threshold is set based on a predetermined separation distance between the distal end of the slave robot and a patient.
(10) The master-slave system according to any one of (1) to (9),
   wherein the slave robot includes a plurality of distal ends, and
   the interference avoidance region is a region excluding a movable region common to the plurality of distal ends of the
(11) The master-slave system according to (10),
   wherein the interference avoidance region includes a movable region for each of the distal ends.
(12) The master-slave system according to any one of (1) to (11),
   wherein, when the height position of the distal end of the slave robot is equal to or higher than a predetermined threshold, the display control unit causes the display unit to display the image.
(13) The master-slave system according to any one of (1) to (12),
   wherein the display control unit causes the display unit to display the image in accordance with an operation of the user on the master robot.
(14) The master-slave system according to any one of (1) to (13),
   wherein a moving speed of the distal end of the slave robot in a case where the height position of the distal end of the slave robot is equal to or higher than a predetermined height position is set to be higher than a moving speed of the distal end of the slave robot in a case where the height position of the distal end of the slave robot is lower than the predetermined height position.
(15) The master-slave system according to any one of (1) to (14), further comprising
   a region setting unit that sets the interference avoidance
(16) The master-slave system according to (15),
   wherein the region setting unit sets the interference avoidance region so that the interference avoidance region is a region where the height position of the distal end of the slave robot is equal to or higher than a predetermined threshold.
(17) The master-slave system according to any one of (1) to (16), further comprising a drive control unit that controls the slave robot so that the distal end of the slave robot moves into the interference avoidance region.
(18) The master-slave system according to (17),
   wherein, when the distal end of the slave robot is located in the interference avoidance region, the drive control unit causes the slave robot to execute a tool replacement operation for replacing a tool of the slave robot with another tool.
(19) A master-slave control device
   causing a display unit that displays an image to a user who operates a master robot to display an image for locating a distal end of a slave robot in an interference avoidance region that has been preset.
(20) A master-slave control method comprising
   a computer causing a display unit that displays an image to a user who operates a master robot to display an image for locating a distal end of a slave robot in an interference avoidance region that has been preset.
(21) A master-slave control device using a component related to the master-slave system according to any one of (1) to (18).
(22) A master-slave control method using a component related to the master-slave system according to any one of (1) to (18).

### REFERENCE SIGNS LIST

1 MASTER-SLAVE SYSTEM
10 MASTER DEVICE
11 MASTER ROBOT
12 ROBOT CONTROL UNIT
13 DISPLAY UNIT
14 COMMUNICATION UNIT
15 SENSOR UNIT
20 SLAVE DEVICE
21 SLAVE ROBOT
22 ROBOT CONTROL UNIT
23 IMAGING UNIT
24 COMMUNICATION UNIT
25 SENSOR UNIT
30 CONTROL DEVICE
31 CONTROL UNIT
32 STORAGE UNIT
33 COMMUNICATION UNIT
111 MASTER ARM
112 FOOT SWITCH
211A SURGICAL TOOL UNIT
211B SURGICAL TOOL UNIT
211a SURGICAL TOOL
211b DRIVE UNIT
212 SLAVE ARM
213 BASE ARM
214 SURGICAL TOOL STAND
214a STORAGE PORTION
311 DRIVE CONTROL UNIT
312 DISPLAY CONTROL UNIT
313 REGION SETTING UNIT
1000 COMPUTER
A10 ICON
A11 ICON
A12 ICON
A13 ICON
A14 ICON
A15 ICON
G1 DISPLAY IMAGE
G10 SURGICAL TOOL SELECTION IMAGE
G11 IMAGE
P1 POINTER
P2 POINTER
R1 VISUAL FIELD
R2 IMAGE
R10 MOVABLE REGION
R20 MOVABLE REGION
R30 COMMON MOVABLE REGION
R100 INTERFERENCE AVOIDANCE REGION
U1 USER
U2 USER
U2a BED

1. A master-slave system comprising:
a display unit that displays an image to a user who operates a master robot; and
a display control unit that causes the display unit to display an image for locating a distal end of a slave robot in an interference avoidance region that has been preset.

2. The master-slave system according to claim 1,
wherein the image is used for guiding the distal end of the slave robot into the interference avoidance region.

3. The master-slave system according to claim 1,
wherein the image is used for generating a trigger for moving the distal end of the slave robot into the interference avoidance region.

4. The master-slave system according to claim 1,
wherein the image is a tool selection image including an icon representing a tool, and
the icon is arranged at a position corresponding to the interference avoidance region in the tool selection image.

5. The master-slave system according to claim 4,
wherein the tool selection image includes a plurality of icons, and
the plurality of icons is arranged at positions corresponding to the interference avoidance region in the tool selection image.

6. The master-slave system according to claim 4,
wherein the display control unit predicts a tool with which replacement is to be performed from a tool of the slave robot and a scene, and displays an icon representing the tool that has been predicted.

7. The master-slave system according to claim 4,
wherein the tool is a surgical, and
the tool selection image is a surgical tool selection image.

8. The master-slave system according to claim 1,
the interference avoidance region is a region where a height position of the distal end of the slave robot is equal to or higher than a predetermined threshold.

9. The master-slave system according to claim 8,
wherein the predetermined threshold is set based on a predetermined separation distance between the distal end of the slave robot and a patient.

10. The master-slave system according to claim 1,
wherein the slave robot includes a plurality of distal ends, and
the interference avoidance region is a region excluding a movable region common to the plurality of distal ends of the slave robot.

11. The master-slave system according to claim 10,
wherein the interference avoidance region includes a movable region for each of the distal ends.

12. The master-slave system according to claim 1, wherein, when the height position of the distal end of the slave robot is equal to or higher than a predetermined threshold, the display control unit causes the display unit to display the image.

13. The master-slave system according to claim 1,
wherein the display control unit causes the display unit to display the image in accordance with an operation of the user on the master robot.

14. The master-slave system according to claim 1,
wherein a moving speed of the distal end of the slave robot in a case where the height position of the distal end of the slave robot is equal to or higher than a predetermined height position is set to be higher than a moving speed of the distal end of the slave robot in a case where the height position of the distal end of the slave robot is lower than the predetermined height position.

15. The master-slave system according to claim 1, further comprising
a region setting unit that sets the interference avoidance region.

16. The master-slave system according to claim 15,
wherein the region setting unit sets the interference avoidance region so that the interference avoidance region is a region where the height position of the distal end of the slave robot is equal to or higher than a predetermined threshold.

17. The master-slave system according to claim 1, further comprising a drive control unit that controls the slave robot so that the distal end of the slave robot moves into the interference avoidance region.

18. The master-slave system according to claim 17,
wherein, when the distal end of the slave robot is located in the interference avoidance region, the drive control unit causes the slave robot to execute a tool replacement operation for replacing a tool of the slave robot with another tool.

19. A master-slave control device
causing a display unit that displays an image to a user who operates a master robot to display an image for locating a distal end of a slave robot in an interference avoidance region that has been preset.

20. A master-slave control method comprising
a computer causing a display unit that displays an image to a user who operates a master robot to display an image for locating a distal end of a slave robot in an interference avoidance region that has been preset.
